(19) United States
(12) Patent Application Publication
(10) Pub. No.: US 2006/0063221 A1
(43) Pub. Date: Mar. 23, 2006

(54) Lanthanide-based substrates and methods for determining clostridial toxin activity

(76) Inventors: Dudley J. Williams, Laguna Niguel, CA (US); Marcella Gilmore, Santa Ana, CA (US); Lance Steward, Irvine, CA (US); Marc Verhagen, Irvine, CA (US); Kei Roger Aoki, Coto de Caza, CA (US)

Correspondence Address: ALLERGAN, INC., LEGAL DEPARTMENT, 2525 DUPONT DRIVE, T2-7H, IRVINE, CA, 92612-1599, US

(21) Appl. No.: 10/947,071

(22) Filed: Sep. 21, 2004

(51) Int. Cl. C12Q 1/37; C12N 11/16

(52) U.S. Cl. 435/23; 435/174

(57) ABSTRACT

The present invention provides a clostridial toxin substrate that contains (a) a lanthanide donor complex; (b) an acceptor having an absorbance spectrum overlapping the emission spectrum of the lanthanide donor complex; and (c) a clostridial toxin recognition sequence containing a cleavage site that intervenes between the lanthanide donor complex and the acceptor, where, under the appropriate conditions, resonance energy transfer is exhibited between the lanthanide donor complex and the acceptor.

### FIELD OF THE INVENTION

The present invention relates generally to protease assays, and more specifically, to methods for determining the presence or activity of clostridial toxins such as botulinum toxins and tetanus toxins using substrates containing lanthanides.

### BACKGROUND INFORMATION

The neuroparalytic syndrome of tetanus and the rare but potentially fatal disease, botulism, are caused by neurotoxins produced by bacteria of the genus Clostridium. These clostridial neurotoxins are highly potent and specific poisons of neural cells, with the human lethal dose of the botulinum toxins on the order of nanograms. Thus, the presence of even minute levels of botulinum toxins in foodstuffs represents a public health hazard that must be avoided through rigorous testing.

However, in spite of their potentially deleterious effects, low controlled doses of botulinum neurotoxins have been successfully used as therapeutics and for some cosmetic applications. In particular, botulinum toxins have been used in the therapeutic management of a variety of focal and segmental dystonias, strabismus, and other conditions in which a reversible depression of cholinergic nerve terminal activity is desired. Established therapeutic uses of botulinum neurotoxins in humans include, without limitation, treatment of blepharospasm, hemifacial spasm, laringeal dysphonia, focal hyperhidrosis, hypersalivation, oromandibular dystonia, cervical dystonia, torticollis, strabismus, limbs dystonia, occupational cramps and myokymia (Rossetto et al., Toxicon 39:27-41 (2001)). As an example, intramuscular injection of spastic tissue with small quantities of botulinum neurotoxin A has been used effectively to treat spasticity due to brain injury, spinal cord injury, stroke, multiple sclerosis and cerebral palsy. Additional possible clinical uses of clostridial neurotoxins are currently being investigated.

Given the potential danger associated with small quantities of botulinum toxins in foodstuffs and the need to prepare accurate pharmaceutical formulations, assays for botulinum neurotoxins presently are employed in the food and pharmaceutical industries. The food industry requires assays for the botulinum neurotoxins to validate new food packaging methods and to ensure food safety. The growing clinical use of the botulinum toxins necessitates accurate assays for botulinum neurotoxin activity for product formulation as well as quality control. In both industries, a mouse lethality test currently is the only acceptable assay for botulinum neurotoxin potency.

Unfortunately, the mouse lethality assay suffers from several drawbacks: cost due to the large numbers of laboratory animals required; lack of specificity; potential for inaccuracy unless large animal groups are used; and sacrifice of animal life. Thus, there is-a need for new methods based on convenient synthetic substrates that can complement and reduce the need for the mouse lethality assay. The present invention satisfies this need by providing novel assays for determining the presence or activity of a clostridial toxin and provides related advantages as well.

### SUMMARY OF THE INVENTION

The present invention provides a clostridial toxin substrate that contains (a) a lanthanide donor complex; (b) an acceptor having an absorbance spectrum overlapping the emission spectrum of the lanthanide donor complex; and (c) a clostridial toxin recognition sequence containing a cleavage site that intervenes between the lanthanide donor complex and the acceptor, where, under the appropriate conditions, resonance energy transfer is exhibited between the lanthanide donor complex and the acceptor.

The present invention further provides a method of determining the presence or activity of a clostridial toxin by (a) treating with a sample, under conditions suitable for clostridial toxin protease activity, a clostridial toxin substrate containing (i) a lanthanide donor complex; (ii) an acceptor having an absorbance spectrum overlapping the emission spectrum of the lanthanide donor complex; and (iii) a clostridial toxin recognition sequence containing a cleavage site that intervenes between the lanthanide donor complex and the acceptor, where, under the appropriate conditions, resonance energy transfer is exhibited between the lanthanide donor complex and the acceptor; (b) exciting an antenna of the lanthanide donor complex; and (c) determining resonance energy transfer of the treated substrate relative to a control substrate, where a difference in resonance energy transfer of the treated substrate as compared to the control substrate is indicative of the presence or activity of the clostridial toxin.

Also provided herein is a nucleic acid molecule which contains a nucleotide sequence encoding a clostridial toxin substrate which includes (a), together with a lanthanide ion, a lanthanide donor complex; (b) an acceptor having an absorbance spectrum overlapping the emission spectrum of the lanthanide donor complex; and (c) a clostridial toxin recognition sequence containing a cleavage site, where the cleavage site intervenes between the lanthanide donor complex and the acceptor and where, under the appropriate conditions, resonance energy transfer is exhibited between the lanthanide donor complex and the acceptor.

### BRIEF DESCRIPTION OF THE DRAWINGS

FIG. 1 shows a schematic of the four steps required for tetanus and botulinum toxin activity in central and peripheral neurons.

FIG. 2 shows the subcellular localization and sites of cleavage of SNAP-25, VAMP and syntaxin. VAMP is bound to synaptic vesicle membrane, whereas SNAP-25 and syntaxin are bound to the target plasma membrane. BoNT/A and /E cleave SNAP-25 close to the carboxy-terminus, releasing nine or 26 residues, respectively. BoNT/B, /D, /F, /G and TeNT act on the conserved central portion of VAMP (dotted) and release the amino-terminal portion of VAMP into the cytosol. BoNT/C1 cleaves SNAP-25 close to the carboxy-terminus as well as cleaving syntaxin at a single site near the cytosolic membrane surface. The action of BoNT/B, /C1, /D, /F, /G and TeNT results in release of a large portion of the cytosolic domain of VAMP or syntaxin, while only a small portion of SNAP-25 is released by selective proteolysis of BoNT/A, /C1 or /E.

FIG. 3 shows an alignment of various SNAP-25 proteins. Human SNAP-25 (SEQ ID NO: 1; GenBank accession g4507099; see, also, related human SNAP-25 sequence g2135800); mouse SNAP-25 (SEQ ID NO: 2; GenBank accession G6755588); Drosophila SNAP-25 (SEQ ID NO: 3; GenBank accession g548941); goldfish SNAP-25 (SEQ ID NO: 4; GenBank accession g2133923); sea urchin SNAP-25 (SEQ ID NO: 5; GenBank accession g2707818) and chicken SNAP-25 (SEQ ID NO: 6; GenBank accession g481202) are depicted.

FIG. 4 shows an alignment of various VAMP proteins. Human VAMP-1 (SEQ ID NO: 7; GenBank accession g135093); human VAMP-2 (SEQ ID NO: 8; GenBank accession g135094); mouse VAMP-2 (SEQ ID NO: 9; GenBank accession g2501081); bovine VAMP (SEQ ID NO: 10; GenBank accession g89782); frog VAMP (SEQ ID NO: 11; GenBank accession g6094391); and sea urchin VAMP (SEQ ID NO: 12; GenBank accession g5031415) are depicted.

FIG. 5 shows an alignment of various syntaxin proteins. Human syntaxin 1A (SEQ ID NO: 13; GenBank accession g15079184), human syntaxin 1B2 (SEQ ID NO: 14; GenBank accession g15072437), mouse syntaxin 1A (SEQ ID NO: 15; GenBank accession g15011853), Drosophila syntaxin 1A (SEQ ID NO: 16; GenBank accession g2501095); C. elegans syntaxin A (SEQ ID NO: 17; GenBank accession g7511662) and sea urchin syntaxin (SEQ ID NO: 18; GenBank accession g13310402) are depicted.

FIG. 6 shows a canonical EF-hand containing an α-helix (E, residues 1-11), a lanthanide-binding loop, and a second α-helix (F, residues 19-29). The α-carbons, indicated by n (residues 2, 5, 6, 9, 17, 22, 25, 26, and (29)) usually have hydrophobic side chains. They point inward and interact with the homologous residues of a second EF-hand domain, related to the first by a local two-fold axis, to form a hydrophobic core. Ile, Leu, or Val at residue 17 attaches the loop to the hydrophobic core. An asterisk indicates variable residues which are often hydrophilic. Gly at position 15 permits a sharp bend in the lanthanide-binding loop. Residues specifically indicated reflect a strong consensus but are not invariant. The lanthanide ion is coordinated by an oxygen atom, or bridging water molecule, of the side chains of residues 10 (X), 12 (Y), 14 (Z), and 18 (—X). The ligand at vertex —Y is the carbonyl oxygen of residue 16. Typically, residue 21 (-Z) is Glu and is the sixth residue to coordinate the lanthanide ion. See Nakayama and Kretsinger, Annu. Rev. Biophys. Biomol. Struct. 23:473-507 (1994).

FIG. 7 shows (A) a schematic of plasmid pQBI GFP-SNAP25₍₁₃₄₋₂₀₆₎₋6XHIS-C and (B) the nucleic acid and amino acid sequences (SEQ ID NOS: 19 and 20) of PQBI GFP-SNAP25₍₁₃₄₋₂₀₆₎-6XHIS-C.

FIG. 8 shows (A) the absorption spectrum and (B) the excitation (dotted) and emission (bold) spectra of GFP-SNAP25₍₁₃₄₋₂₀₆₎-His6C.

FIG. 9 shows (A) the UV-VIS absorption spectrum and (B) the emission spectrum using pulse gated excitation at 300 nm of GFP-SNAP25₍₁₃₄₋₂₀₆₎-His6-C—CS124-DTPA-EMCH—Tb.

FIG. 10 shows a luminescence resonance energy transfer (LRET) assay of clostridial toxin activity using the lanthanide-based substrate GFP-SNAP25₍₁₃₄₋₂₀₆₎-His6-C—CS124-DTPA-EMCH—Tb. (A) Quench relief shown by LRET upon addition of dilute reduced bulk BoNT/A at 131 ng/ml cuvette concentration at 37° C. The terbium emission at 586 nm increased upon addition of toxin. (B) Emission spectrum of GFP-SNAP25₍₁₃₄₋₂₀₆₎-His6-C—CS124-DTPA-EMCH—Tb using pulse gated Xenon excitation at 330 nm before and after turnover. The dotted trace represents gated terbium emission before turnover while the solid trace represents gated terbium emission after turnover.

### DETAILED DESCRIPTION OF THE INVENTION

The invention provides novel substrates and methods for determining the presence or activity of clostridial toxins including botulinum toxins of all serotypes as well as tetanus toxins. The novel methods of the invention, which rely on a clostridial toxin substrate containing a lanthanide ion such as terbium, reduce the need for animal toxicity studies and can be used to analyze crude and bulk samples as well as highly purified dichain or single chain toxins or formulated toxin products. The novel lanthanide-based methods of the invention can be performed as homogeneous solution-phase assays and are amenable to automated high-throughput formats. Furthermore, the methods of the invention can be performed as time-resolved assays, which are particularly useful in analyzing samples containing non-specific background fluorescence.

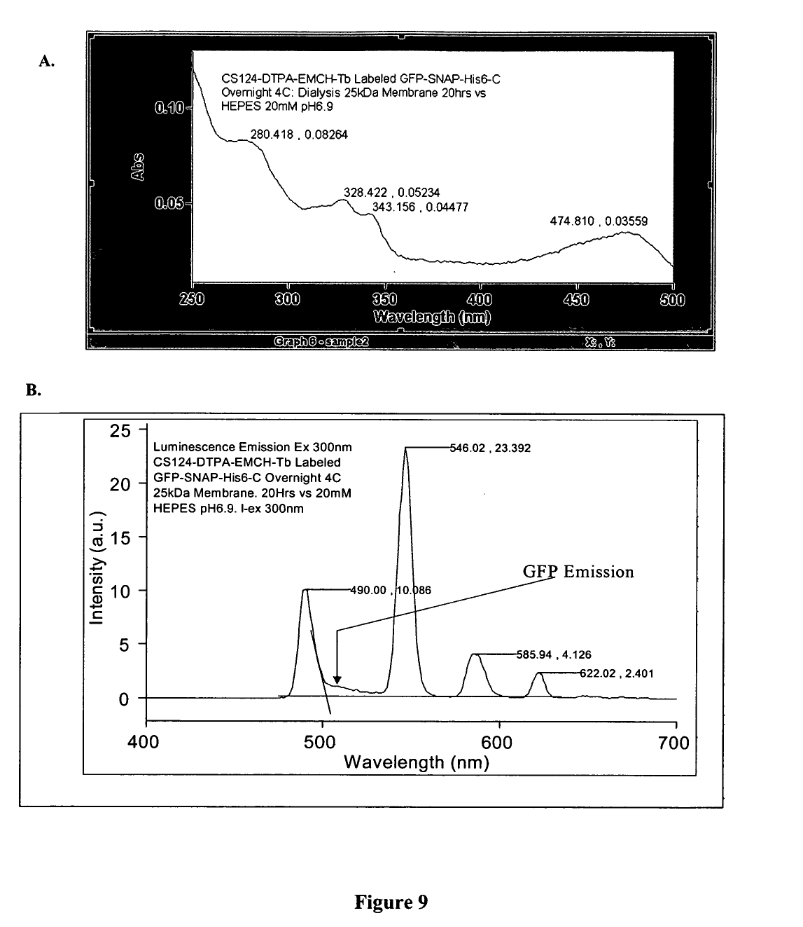

As disclosed herein in Example I, a recombinant fusion protein was prepared containing green fluorescent protein fused to a portion of SNAP-25 and further engineered to contain a carboxy-terminal cysteine. Maleimide chemistry was used to derivatize the carboxy-terminal cysteine of GFP-SNAP25₍₁₃₄₋₂₀₆₎-His6-C with the lumiphore CS124-DTPA-EMCH—Tb. The absorption and emission spectra of the CS124-DTPA-EMCH—Tb labeled GFP-SNAP25₍₁₃₄₋₂₀₆₎-His6-C are shown in FIGS. 9A and 9B, respectively. As can be seen in FIG. 9B, excitation of the sensitizing group carbostyryl 124 (CS124) at 330 nm resulted in the characteristic long lifetime emission of terbium which yields a series of four prominent sharp bands at 490 nm, 546 nm, 586 nm and 622 nm.

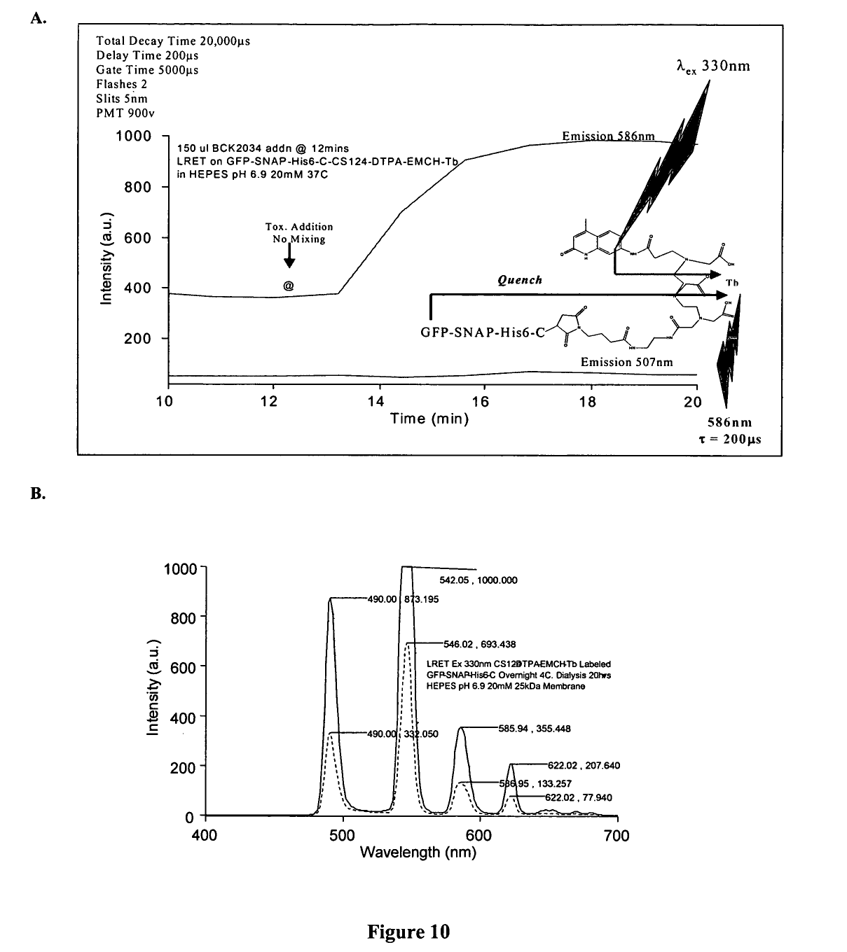

As further disclosed herein in Example II, this clostridial toxin substrate was useful for sensitively assaying for the activity of bulk BoNT/A toxin. In particular, energy transfer between the lanthanide donor complex and GFP was observed by monitoring terbium emission at 586 nm. As shown in FIG. 10A, there was a notable increase in luminescence intensity at 586 nm following addition of reduced bulk BoNT-A toxin, indicative of the relief of quenching between the lanthanide donor complex and GFP. Furthermore, the signal to noise ratio for the emission process was greatly enhanced by utilizing a gated process to monitor the emission as shown in FIG. 10B, in which the solid trace represents gated terbium emission before turnover of substrate and the dotted trace represents gated terbium emission after turnover.

In sum, these results indicate that GFP-SNAP25₍₁₃₄₋₂₀₆₎-His6-C can be derivatized with a commercially available lanthanide donor complex such as CS124-DTPA-EMCH—Tb to produce a clostridial toxin substrate which exhibits quenching between the lanthanide donor complex and GFP. The relief of quenching, as indicated by an increase in luminescence intensity upon addition of the clostridial toxin is indicative of the presence or activity of the clostridial toxin. These results further indicate that the use of gated emission can be useful for reducing background when assaying for clostridial toxin activity with a lanthanide-based substrate of the invention.

Based on these findings, the present invention provides a clostridial toxin substrate which contains (a) a lanthanide donor complex; (b) an acceptor having an absorbance spectrum overlapping the emission spectrum of the lanthanide donor complex; and (c) a clostridial toxin recognition sequence containing a cleavage site that intervenes between the lanthanide donor complex and the acceptor, where, under the appropriate conditions, resonance energy transfer is exhibited between the lanthanide donor complex and the acceptor. In one embodiment, the invention provides a clostridial toxin substrate which includes a lanthanide donor complex having a fluorescence lifetime of at least 500 μs. In another embodiment, the invention provides a clostridial toxin substrate which includes a lanthanide donor complex having a fluorescence quantum yield of at least 0.05. In still another embodiment, the invention provides a clostridial toxin substrate which includes a lanthanide donor complex having a fluorescence quantum yield of at least 0.5.

Lanthanide ions useful in a lanthanide donor complex encompass, without limitation, terbium ions, europium ions, samarium ions and dysprosium ions. Lanthanide-binding sites useful in a lanthanide donor complex can have, for example, an affinity for a lanthanide ion of at least 5 μM and include, but are not limited to, peptides and peptidomimetics such as, without limitation, those including the coordination site of an EF hand motif or including an EF hand motif. Lanthanide-binding sites useful in a lanthanide donor complex further include, yet are not limited to, thiol-reactive chelators; diethylenetriaminepentacetic acid (DTPA); β-diketone chelates; polyaminopolycarboxylic acid chelates; calixarene chelates; polyphenol; DOTA; pyridine and polypyridine. Additional lanthanide-binding sites useful in the invention include, without limitation, trisbipyridine (TBP) cryptates; trisbipyridine tetracarboxylate (TBP4COOH) cryptates; trisbipyridine pentacarboxylate (TBP5COOH) cryptates; and pyridine bipyridine tetracarboxylates (PBP4COOH).

A lanthanide donor complex includes an antenna which can be distinct from, or incorporated within, the lanthanide-binding site of the donor complex. An antenna useful in the invention can be, without limitation, carbostyryl124 (CS124), tryptophan, or 2-hydroxyisophthalamide. In one embodiment, the invention provides a clostridial toxin substrate incorporating a lanthanide donor complex which includes carbostyryl124 (CS124) as the antenna. In another embodiment, the invention provides a clostridial toxin substrate in which the lanthanide donor complex is CS124-DTPA-EMCH—Tb.

A variety of acceptors are useful in the clostridial toxin substrates of the invention including, without limitation, acceptor fluorophores such as Alexa Fluor dyes and other non-protein acceptors. Acceptor fluorophores useful in the invention further include, such as green fluorescent protein (GFP), blue fluorescent protein (BFP), yellow fluorescent protein (YFP), cyan fluorescent protein (CFP) and red fluorescent protein (RFP). In one embodiment, the invention provides a clostridial toxin substrate which includes green fluorescent protein as the acceptor. Non-fluorescent acceptors also are useful in the clostridial toxin substrates of the invention and include, without limitation, heme proteins.

A variety of recognition sequences can be included in a clostridial toxin substrate of the invention. In one embodiment, the recognition sequence is a BoNT/A recognition sequence such as, without limitation, a BoNT/A recognition sequence containing at least six consecutive residues of SNAP-25, where the six consecutive residues include Gln-Arg, or a peptidomimetic thereof. Such a BoNT/A recognition sequence can include, for example, residues 134 to 206 of SEQ ID NO: 2. A recognition sequence included in a clostridial toxin substrate of the invention also can be, without limitation, a BoNT/B recognition sequence. Such a BoNT/B recognition sequence can contain, for example, at least six consecutive residues of VAMP, where the six consecutive residues include Gln-Phe, or a peptidomimetic thereof. In a further embodiment, a recognition sequence included in a clostridial toxin substrate is a BoNT/C1 recognition sequence. Such a BoNT/C1 recognition sequence can contain, without limitation, at least six consecutive residues of syntaxin, where the six consecutive residues include Lys-Ala, or a peptidomimetic thereof. A BoNT/C1 recognition sequence useful in the invention also can contain at least six consecutive residues of SNAP-25, where the six consecutive residues include Arg-Ala, or a peptidomimetic thereof.

In a further embodiment, a recognition sequence included in a clostridial toxin substrate is a BoNT/D recognition sequence. Such a BoNT/D recognition sequence can contain, for example, at least six consecutive residues of VAMP, where the six consecutive residues include Lys-Leu, or a peptidomimetic thereof. A recognition sequence useful in the invention also can be, for example, a BoNT/E recognition sequence. Such a BoNT/E recognition sequence can contain, without limitation, at least six consecutive residues of SNAP-25, where the six consecutive residues include Arg-Ile, or a peptidomimetic thereof. In yet another embodiment, a recognition sequence included in a clostridial toxin substrate of the invention is a BoNT/F recognition sequence. BoNT/F recognition sequences useful in the invention encompass, without limitation, those having at least six consecutive residues of VAMP, where the six consecutive residues include Gln-Lys, or a peptidomimetic thereof. A recognition sequence included in a clostridial toxin substrate also can be a BoNT/G recognition sequence. Such BoNT/G recognition sequences encompass, without limitation, those having at least six consecutive residues of VAMP, where the six consecutive residues include Ala-Ala, or a peptidomimetic thereof. In still a further embodiment, a recognition sequence included in a clostridial toxin substrate of the invention is a tetanus toxin (TeNT) recognition sequence. Such a TeNT recognition sequence can be, without limitation, a sequence containing at least six consecutive residues of VAMP, where the six consecutive residues include Gln-Phe, or a peptidomimetic thereof.

A clostridial toxin substrate of the invention can be, without limitation peptide or peptidomimetic, which can have any of a variety of lengths. In particular embodiments, a clostridial toxin substrate of the invention is a peptide or peptidomimetic having at most 300 residues or at most 150 residues. A clostridial toxin substrate of the invention can be cleaved with a range of activities. In one embodiment, a clostridial toxin substrate of the invention can be cleaved with an activity of at least 1 nanomole/minute/milligram toxin. In another embodiment, a clostridial toxin substrate of the invention can be cleaved with an activity of at least 20 nanomoles/minute/milligram toxin. In a further embodiment, a clostridial toxin substrate of the invention can be cleaved with an activity of at least 100 nanomoles/minute/milligram toxin.

The present invention further provides a nucleic acid molecule which contains a nucleotide sequence encoding a clostridial toxin substrate which includes (a), together with a lanthanide ion, a lanthanide donor complex; (b) an acceptor having an absorbance spectrum overlapping the emission spectrum of the lanthanide donor complex; and (c) a clostridial toxin recognition sequence containing a cleavage site, where the cleavage site intervenes between the lanthanide donor complex and the acceptor and where, under the appropriate conditions, resonance energy transfer is exhibited between the lanthanide donor complex and the acceptor. A nucleic acid molecule of the invention can encode a clostridial toxin substrate with any of a variety of lengths; in particular embodiments, a nucleic acid molecule of the invention encodes a clostridial toxin substrate having a length of at most 300 residues, or a length of at most 150 residues.

A lanthanide donor complex includes, in part, a lanthanide-binding site. Any of a variety of lanthanide-binding sites are useful in the invention including, without limitation, those which contain the coordination site of an EF hand motif and those which include an EF hand motif. In some embodiments, a lanthanide donor complex includes a tryptophan reisdues which acts as an antenna. In another embodiment, a nucleic acid molecule of the invention encodes a clostridial toxin substrate in which the acceptor is an acceptor fluorophore. In still further embodiments, a nucleic acid molecule of the invention encodes a clostridial toxin substrate in which the acceptor fluorophore is green fluorescent protein (GFP), blue fluorescent protein (BFP), yellow fluorescent protein (YFP), cyan fluorescent protein (CFP) or red fluorescent protein (RFP). In yet another embodiment, a nucleic acid molecule of the invention encodes a clostridial toxin substrate in which the acceptor is a non-fluorescent acceptor such as, without limitation, a heme protein.

A clostridial toxin substrate encoded by a nucleic acid molecule of the invention can include any of a variety of recognition sequences. In a nucleic acid molecule of the invention, the encoded recognition sequence can be, for example, a BoNT/A recognition sequence such as, without limitation, a BoNT/A recognition sequence containing at least six consecutive residues of SNAP-25, where the six consecutive residues include Gln-Arg, or a peptidomimetic thereof. Such a BoNT/A recognition sequence can include, for example, residues 134 to 206 of SEQ ID NO: 2. An encoded recognition sequence useful in a nucleic acid molecule of the invention also can be, without limitation, a BoNT/B recognition sequence. Such a BoNT/B recognition sequence can contain, for example, at least six consecutive residues of VAMP, where the six consecutive residues include Gln-Phe, or a peptidomimetic thereof. In a further embodiment, a nucleic acid molecule of the invention encodes a clostridial toxin substrate which includes a BoNT/C1 recognition sequence. Such a BoNT/C1 recognition sequence can contain, without limitation, at least six consecutive residues of syntaxin, where the six consecutive residues include Lys-Ala, or a peptidomimetic thereof. A BoNT/C1 recognition sequence useful in the invention also can contain at least six consecutive residues of SNAP-25, where the six consecutive residues include Arg-Ala, or a peptidomimetic thereof.

In a further embodiment, a nucleic acid molecule of the invention encodes a clostridial toxin substrate which includes a BoNT/D recognition sequence. Such a BoNT/D recognition sequence can contain, for example, at least six consecutive residues of VAMP, where the six consecutive residues include Lys-Leu, or a peptidomimetic thereof. In another embodiment, a nucleic acid molecule of the invention encodes a clostridial toxin substrate which includes a BoNT/E recognition sequence. Such a BoNT/E recognition sequence can contain, without limitation, at least six consecutive residues of SNAP-25, where the six consecutive residues include Arg-Ile, or a peptidomimetic thereof. In yet another embodiment, a nucleic acid molecule of the invention encodes a clostridial toxin substrate which includes a BoNT/F recognition sequence. BoNT/F recognition sequences useful in the invention encompass, without limitation, those having at least six consecutive residues of VAMP, where the six consecutive residues include Gln-Lys, or a peptidomimetic thereof. A nucleic acid molecule of the invention also can encode a clostridial toxin substrate which has a BoNT/G recognition sequence. Such a BoNT/G recognition sequence can be, for example, one having at least six consecutive residues of VAMP, where the six consecutive residues include Ala-Ala, or a peptidomimetic thereof. In another embodiment, a nucleic acid molecule of the invention encodes a clostridial toxin substrate which includes a TeNT recognition sequence. Such a TeNT recognition sequence can be, without limitation, a sequence containing at least six consecutive residues of VAMP, where the six consecutive residues include Gln-Phe, or a peptidomimetic thereof.

The tetanus and botulinum neurotoxins which can be assayed using a substrate or method of the invention are produced by Clostridia. These toxins cause the neuroparalytic syndromes of tetanus and botulism, with tetanus toxin acting mainly within the central nervous system and botulinum toxin acting on the peripheral nervous system. Clostridial neurotoxins share a similar mechanism of cell intoxication in which the release of neurotransmitters is blocked. In these toxins, which are composed of two disulfide-linked polypeptide chains, the larger subunit is responsible for neurospecific binding and translocation of the smaller subunit into the cytoplasm. Upon translocation and reduction in neurons, the smaller chain displays peptidase activity specific for protein components involved in neuroexocytosis. The “SNARE” protein targets of clostridial toxins are common to exocytosis in a variety of non-neuronal types; in these cells, as in neurons, light chain peptidase activity inhibits exocytosis.

Tetanus neurotoxin and botulinum neurotoxins B, D, F, and G specifically recognize VAMP (also known as synaptobrevin), an integral protein of the synaptic vesicle membrane. VAMP is cleaved at distinct bonds depending on the neurotoxin. Botulinum A and E neurotoxins recognize and cleave specifically SNAP-25, a protein of the presynaptic membrane, at two different sites in the carboxy-terminal portion of the protein. Botulinum neurotoxin C cleaves syntaxin, a protein of the nerve plasmalemma, in addition to SNAP-25. The three protein targets of the Clostridial neurotoxins are conserved from yeast to humans although cleavage sites and toxin susceptibility are not necessarily conserved (see below; see, also, Humeau et al., Biochimie 82:427-446 (2000); Niemann et al., Trends in Cell Biol. 4:179-185 (1994); and Pellizzari et al., Phil. Trans. R. Soc. London 354:259-268 (1999)).

Naturally occurring tetanus and botulinum neurotoxins are produced as polypeptide chains of 150 kDa without a leader sequence. These toxins may be cleaved by bacterial or tissue proteinases at an exposed protease-sensitive loop, generating dichain toxin. Selective proteolytic cleavage activates the toxins by generating two disulfide-linked chains: an L chain of 50 kDa and an H chain of 100 kDa, which is composed of two domains denoted H_{N} and H_{C}. This dichain toxin is substantially more active than the unnicked toxin. Naturally occurring clostridial toxins contain a single interchain disulfide bond bridging the heavy chain and light chain; such a bridge is important for neurotoxicity of toxin added extracellularly (Montecucco and Schiavo, Quarterly Rev. Biophysics 28:423-472 (1995)).

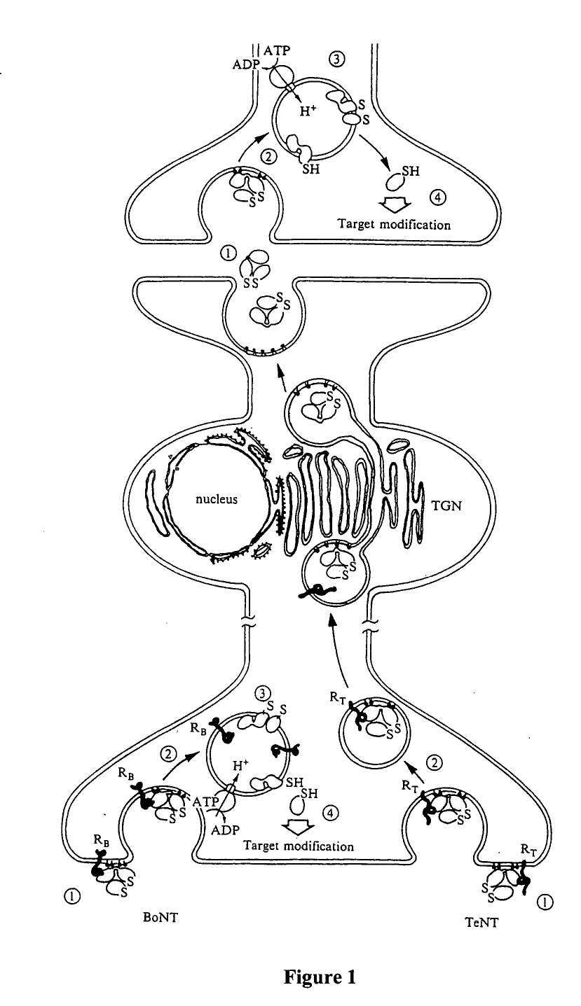

The clostridial toxins appear to be folded into three distinct domains of about 50 kDa which are connected by loops, with each domain having a distinct functional role. As illustrated in FIG. 1, the cell intoxication mechanism of the clostridial toxins consists of four distinct steps: (1) binding; (2) internalization; (3) membrane translocation; and (4) enzymatic target modification. The carboxy-terminal domain of the heavy chain (H_{C}) functions in neurospecific binding, while the amino-terminal domain of the H chain (H_{N}) functions in membrane translocation from endosome to cell cytoplasm. Following reduction of the disulfide linkage inside the cell, the zinc-endopeptidase activity of the L chain is liberated (Montecucco and Schiavo, supra, 1995).

The amino acid sequences of eight human clostridial neurotoxin serotypes have been derived from the corresponding genes (Niemann, “Molecular Biology of Clostridial Neurotoxins” in Sourcebook of Bacterial Protein Toxins Alouf and Freer (Eds.) pp. 303-348 London: Academic Press 1991). The L chain and H chain are composed of roughly 439 and 843 residues, respectively. Homologous segments are separated by regions of little or no similarity. The most well conserved regions of the L chain are the amino-terminal portion (100 residues) and central region (corresponding to residues 216 to 244 of TeNT), as well as the two cysteines forming the interchain disulfide bond. The 216 to 244 region contains a His-Glu-X—X-His binding motif characteristic of zinc-endopeptidases.

The clostridial toxin heavy chains are less well conserved than the light chains, with the carboxy-terminal portion of H_{C} corresponding to residues 1140 to 1315 of TeNT the most variable. This is consistent with the involvement of the H_{C} domain in binding to nerve terminals and the fact that different neurotoxins appear to bind different receptors.

Comparison of the nucleotide and amino acid sequences of the clostridial toxins indicates that they derive from a common ancestral gene. Spreading of these genes may have been facilitated by the fact that the clostridial neurotoxin genes are located on mobile genetic elements. As discussed further below, sequence variants of the seven botulinum toxins are known in the art. See, for example, Humeau et al., supra, 2000.

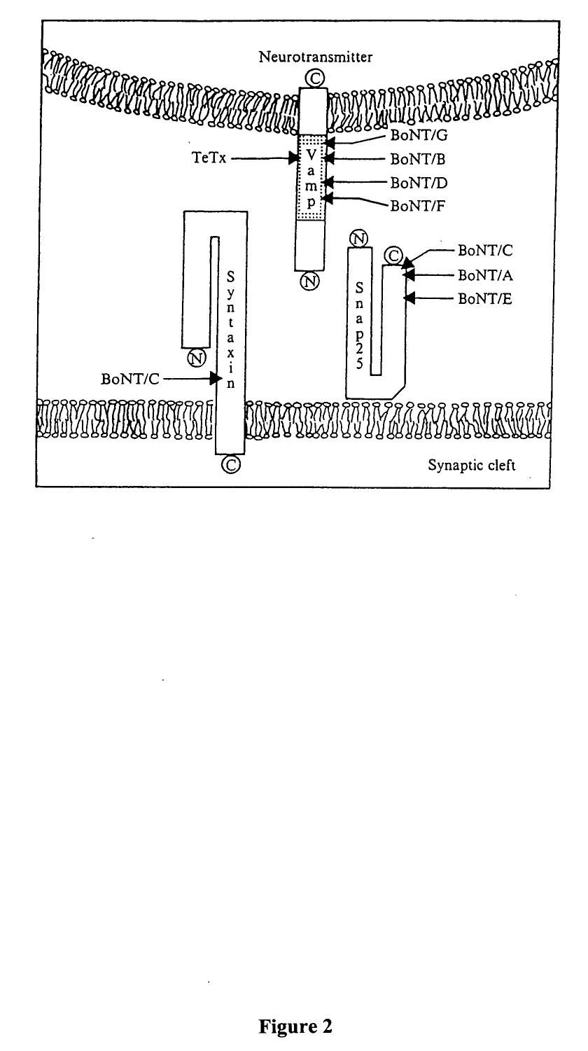

As discussed above, natural targets of the clostridial neurotoxins include VAMP, SNAP-25, and syntaxin. VAMP is associated with the synaptic vesicle membrane, whereas SNAP-25 and syntaxin are associated with the target membrane (see FIG. 2). BoNT/A and BoNT/E cleave SNAP-25 in the carboxy-terminal region, releasing nine or twenty-six amino acid residues, respectively, and BoNT/C1 also cleaves SNAP-25 near the carboxy-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. Thus, BoNT/B, BoNT/C1, BoNT/D, BoNT/F, BoNT/G or TeNT proteolysis results in release of a large portion of the cytosolic domain of VAMP or syntaxin, while only a small portion of SNAP-25 is released by BoNT/A, BoNT/C1 or BoNT/E cleavage (Montecucco and Schiavo, supra, 1995).

Naturally occurring SNAP-25, a protein of about 206 residues lacking a transmembrane segment, is associated with the cytosolic surface of the nerve plasmalemma (FIG. 2; see, also, Hodel et al., Int. J. Biochemistry and Cell Biology 30:1069-1073 (1998)). In addition to homologs highly conserved from Drosophila to mammals, SNAP-25-related proteins also have been cloned from yeast. SNAP-25 is required for axonal growth during development and may be required for nerve terminal plasticity in the mature nervous system. In humans, two isoforms are differentially expressed during development; isoform a is constitutively expressed during fetal development, while isoform b appears at birth and predominates in adult life. SNAP-25 analogues such as SNAP-23 also are expressed outside the nervous system, for example, in pancreatic cells.

Naturally occurring VAMP is a protein of about 120 residues, with the exact length depending on the species and isotype. As shown in FIG. 2, VAMP contains a short carboxy-terminal segment inside the vesicle lumen while most of the molecule is exposed to the cytosol. The proline-rich amino-terminal thirty residues are divergent among species and isoforms while the central portion of VAMP (residues 30 to 96), which is rich in charged and hydrophilic residues and includes known cleavage sites, is highly conserved. VAMP colocalizes with synaptophysin on synaptic vesicle membranes.

A variety of species homologs of VAMP are known in the art including human, rat, bovine, Torpedo, Drosophila, yeast, squid and Aplysia homologs. In addition, multiple isoforms of VAMP have been identified including VAMP-1, VAMP-2 and cellubrevin, and forms insensitive to toxin cleavage have been identified in non-neuronal cells. VAMP appears to be present in all vertebrate tissues although the distribution of VAMP-1 and VAMP-2 varies in different cell types. Chicken and rat VAMP-1 are not cleaved by TeNT or BoNT/B. These VAMP-1 homologs have a valine in place of the glutamine present in human and mouse VAMP-1 at the TeNT or BoNT/B cleavage site. The substitution does not affect BoNT/D, /F or /G, which cleave both VAMP-1 and VAMP-2 with similar rates.

Syntaxin is located on the cytosolic surface of the nerve plasmalemma and is membrane-anchored via a carboxy-terminal segment, with most of the protein exposed to the cytosol. Syntaxin colocalizes with calcium channels at the active zones of the presynaptic membrane, where neurotransmitter release takes place. In addition, syntaxin interacts with synaptotagmin, a protein of the SSV membrane, that forms a functional bridge between the plasmalemma and the vesicles. A variety of syntaxin isoforms have been identified. Two isoforms of slightly different length (285 and 288 residues) have been identified in nerve cells (isoforms 1A and 1B), with isoforms 2, 3, 4 and 5 expressed in other tissues. The different isoforms have varying sensitivities to BoNT/C1, with the 1A, 1B, 2 and 3 syntaxin isoforms cleaved by this toxin.

The lanthanides, or “rare earth” elements, are a group of elements whose trivalent cations emit light at well-defined wavelengths and with long decay times. Lanthanides include, without limitation, elements with atomic numbers 57 through 71: lanthanide (La); cerium (Ce); praseodymium (Pr); neodymium (Nd); promethium (Pm); samarium (Sm); europium (Eu); gadolinium (Gd); terbium (Tb); dysprosium (Dy); holmium (Ho); erbium (Er); thulium (Tm); ytterbium (Yb); and lutetium (Lu). Lanthanides can further include, without limitation, yttrium (Y; atomic number 39) and scandium (Sc; atomic number 21).

Lanthanide ions have unique photophysical and spectral properties based on their special electronic configuration which partly shields optically active electrons. The emission lifetimes of the lanthanide ions are usually long; however, their light collection efficiency is very poor. Given these properties, lanthanide ions are particularly useful in conjunction with a light-harvesting device (“antenna”), which can be, for example, a strongly absorbing aromatic chromophore such as a pyridyl, phenyl or indole group. The energy collected by the antenna is transferred by intramolecular non-radiative processes from the singlet to the triplet state of the moiety, then from the triplet to the emissive level of the lanthanide ion, which subsequently emits its characteristic long-lived luminescence. Thus, a lanthanide ion in conjunction with an antenna is useful as a luminescent probe, for example, in highly sensitive time-resolved assays, where it generates a long-lived fluorescent signal that can be readily distinguished from short-lived background fluorescence present in many biological samples.

Lanthanides generally exist as trivalent cations, in which case their electronic configuration is (Xe)4fⁿ, with n varying from 1 (Ce³⁺) to 14 (Lu³⁺). Without wishing to be bound by the following, the transitions of the f-electrons can be responsible for the special photophysical properties of the lanthanide ions such as long-lived luminescence and sharp absorption and emission lines. In particular, f-electrons can be shielded from external perturbations by filled 5s and 5p orbitals, resulting in characteristic line-like spectra. f-f electronic transitions are forbidden, leading to long excited state lifetimes in the microsecond to millisecond range.

As discussed above, in many cases energy can be transferred to a lanthanide ion from a nearby organic chromophore, known as an “antenna” or “sensitizer.” Thus, a lanthanide donor complex useful in the invention includes a lanthanide ion, a lanthanide-binding site and an antenna and generally is structured to shield the lanthanide ion from the quenching effects of water or other solvent. The lanthanide-binding site functions to retain the lanthanide ion and may optionally act as a scaffold for attachment of an antenna and a reactive group suitable for coupling the lanthanide donor complex to the remainder of the clostridial toxin substrate. In one embodiment, the antenna is incorporated within the lanthanide-binding site. In another embodiment, an antenna separate from the lanthanide-binding site is included in the lanthanide donor complex.

Lanthanide ions useful in the invention include, without limitation, terbium (Tb), europium (Eu), dysprosium (Dy) and samarium (Sm) ions, which are lanthanides that emit in the visible spectra. In one embodiment, a lanthanide ion is a Tb or Eu ion, which has a high emission quantum yield and emits with stronger intensity than a Dy or Sm ion. Excitation of an antenna for Tb or Eu is in the ultraviolet range and can be achieved, for example, using a nitrogen laser at 337 nm, or a flash lamp. Terbium emission is in the green spectra, while europium emission is in the red spectra, both providing a contrast to the excitation light.

As used herein, the term “antenna” is synonymous with “sensitizer” and means a molecule such as an organic chromophore which absorbs excitation light and transfers the light energy to a lanthanide ion. An antenna is necessary because of the inherently weak absorbance of lanthanide ions themselves. In one embodiment, the antenna is carbostyril124 (CS124), which absorbs light with an excitation of 337 nm. In another embodiment, the antenna is a tryptophan residue. In a further embodiment, the antenna is 2-hydroxyisophthalamide, which also acts as a lanthanide-binding site (see below). It is understood that an antenna can be distinct from, or can make up part of a lanthanide binding-site. As non-limiting examples, an antenna which binds a lanthanide ion can be 2-hydroxyisophthalamide, a pyridine or other cryptate; a LANCE complex (Wallac; Perkin-Elmer); or a terpyridine complex.

As used herein, the term “lanthanide-binding site” means a moiety that constrains a lanthanide ion. A variety of lanthanide-binding sites are useful in the clostridial toxin substrates of the invention. Exemplary classes of lanthanide-binding sites include, but are not limited to, polyaminopolycarboxylic acid chelates such as DTPA chelates, BPTA chelates, β-diketone chelates, pyridines, polypyridines and calixarene chelates. These and other lanthanide chelates are known in the art as described in Li and Selvin, Bioconj. Chem. 8:127-132 (1997); Chen and Selvin, Bioconj. Chem. 10: 311-315 (1999); Selvin, Nature Struc. Biol. 7:730-734 (2000); Selvin, Methods Enzym. 246:300-334 (1995); Selvin et al., J. Am. Chem. Soc. 116:6029-6030 (1994); and Yuan et al., Anal. Chem. 73:1869-1876 (2001). In one embodiment, a lanthanide-binding site useful in the invention is a polyaminocarboxylate such as diethylenetriaminepentacetic acid (DTPA) or triethylenetetraaminehexaacetic acid (TTHA). An antenna which is useful in conjunction with a polyaminocarboxylate lanthanide-binding site such as DTPA or TTHA can be, without limitation, carbostyril124 (CS124).

Lanthanide-binding sites useful a lanthanide donor complex include those which are peptides and peptidomimetics. In one embodiment, a lanthanide-binding site useful in the invention includes the coordination site of an EF hand motif, which is a highly conserved domain in which two helices enclose a binding loop with high affinity for Ca²⁺, Tb³⁺ and other ions with similar ionic radii. In nature, more than 200 proteins including calmodulin, troponin C, parvalbumin and calbindin contain one or several copies of an EF hand.

In nature, the two α-helices of an EF hand motif are connected by a loop of about 12 residues which contains the metal coordination site of the motif. The residues which serve as ligands are highly conserved within a contiguous sequence of twelve residues spanning the loop and the beginning of the second helix. In particular, the residues at positions 1, 3, 5, 7, 9, and 12 of this loop region and possibly a coordinating water molecule provide seven coordination oxygens for the lanthanide ion. Acidic amino acids are frequently present at most or all of the coordinating positions with the exception of Trp at position 7, where the coordination oxygen is provided by the main chain (Vasquez-Ibar et al., Proc. Natl. Acad. Sci. USA 99:3487-3492 (2002)). Loop residues in positions 1, 3, 5 and 12 contribute monodentate (positions 1, 3 and 5) or bidentate (position 12) ligands through side chain oxygens; residue 7 (tryptophan) ligands through its backbone carbonyl oxygen. An invariant glycine residue is present at position 6 to allow the sharp bend necessary to ligate the lanthanide through the oxygen of residue 5 and the carbonyl of residue 7. In addition, residue 9 provides a ligand either directly though an oxygen of its side chain or indirectly via a water molecule. Residue 12 is an invariant glutamic acid (Glu), while residue 1 is typically aspartate (Asp). See Lewit-Bentley, Curr. Opin. Struct. Biol. 10:637-643 (2000); and Myers (Ed.), Molecular Biology and Biotechnology VCH publishers New York, N.Y. (1995).

As used herein, the term “coordination site of an EF hand motif” means a sequence of about 12 residues in which position 6 is a glycine; position 12 is a glutamic acid, and ligand groups at positions 1, 3, 5, 7, 9, and 12 of the sequence, or a coordinating water molecule, provide a metal binding site. A tryptophan residue optionally can be present at position 7. It is understood that a lanthanide-binding site which includes the coordination site of an EF hand motif may or may not have homology to the α-helices of an EF hand motif outside the 12 residue coordination site.

A sequence which includes the coordination site of an EF hand motif can be, for example, the 14-mer peptide GDKNADGWIEFEEL (SEQ ID NO: 97) as described in MacManus et al., Biosci. Rep. 3:1071-1075 (1983), and Strynadka and James, Annu. Rev. Biochem. 58: 951-998 (1989). The 14-mer SEQ ID NO: 97 functions as both a lanthanide-binding site and an antenna due to the inclusion of a tryptophan residue. Coordination sites of an EF hand motif further include, without limitation, the peptide GDKNADGFICFEEL (SEQ ID NO: 98), where the indicated cysteine residue can be covalently labeled with iodoacetamidosalicylic acid or another antenna (Clark et al., FEBS 333: 96-98 (1993)), and the peptide DKNADGCIEFEE (SEQ ID NO: 99), where the indicated cysteine residue permits convenient covalent attachment of an antenna (Clark et al., Anal. Biochem. 210:1-6 (1993)). As non-limiting examples, 7-diethylamino-3-((4′-iodoacetylamino)phenyl)-4-methylcoumarin can be covalently attached to the cysteine in SEQ ID NO: 99, for example, as an antenna for Eu³⁺, and 4-iodoacetamidosalicylic acid can be covalently attached to the cysteine in SEQ ID NO: 99, for example, as an antenna for Tb³⁺.

A lanthanide-binding site which includes the coordination site of an EF hand motif also can be a lanthanide-binding tag (LBT) such as one described in Nitz et al., Angew. Chem. Int. Ed. 43:3682-3685 (2004)). Such a lanthanide-binding site can include, without limitation, the 17-mer YID₁TN₃ND₅GW₇YE₉GDE₁₂LLA (SEQ ID NO: 100), which includes the antenna tryptophan. Such a lanthanide-binding site can, for example, coordinate a terbium or other lanthanide ion through eight ligands, in particular, monodentate oxygen ligands of Asp1, Asn3 and Asp5, bidentate ligands from Glu9 and Glu12, and the backbone carbonyl of Trp 7. Furthermore, lanthanide-binding sites such as those described in Nitz et al., supra, 2004, can bind a terbium or other lanthanide ion with nanomolar affinities. As non-limiting examples, the lanthanide-binding site SEQ ID NO: 100 binds Eu³⁺ with an apparent dissociation constant Kd of 62±4 nM; Gd³⁺ with an apparent dissociation constant Kd of 84±6 nM; Tb³⁺ with an apparent dissociation constant Kd of 57±3 nM; Dy³⁺ with an apparent dissociation constant Kd of 71±5 nM; and Er³⁺ with an apparent dissociation constant Kd of 78±6 nM.

Lanthanide-binding sites useful in a lanthanide donor complex further include those which bind a lanthanide ion exclusively through peptide-based ligands, excluding water molecules from the lanthanide ion coordination sphere. Such a lanthanide-binding site can include, for example, the 17-mer sequence YIDTNN DGWYEGDELLA (SEQ ID NO: 100; Nitz et al., supra, 2004).

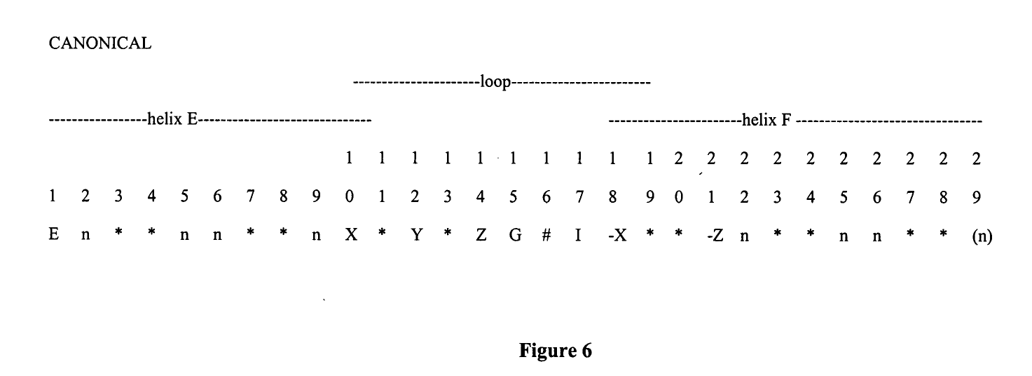

A lanthanide-binding site useful in a lanthanide donor complex also can be an EF hand motif. As used herein, the term “EF hand motif” means two α-helices flanking the coordination site of an EF hand motif. An EF hand motif useful in the invention can be, without limitation, an EF hand from one of the following subfamilies: calmodulin (CAM); troponin C (TNC); essential or regulatory light chain of myosin; troponin, nonvertebrate (TPNV); Call, C. elegans (CAL); squidulin, Loligo (SQUD); CDC31 and caltractin (CDC); calcium-dependent protein kinase (CDPK); LAV1, Physarum (LAV); EHF5; calcineurin B (CLNB); p24 thyroid protein, Canis (TPP); calbindin 28 kDa (CLBN); parvalbumin (PARV); intestinal calcium binding protein and S100; diacylglycerol kinase (DGK); α-actinin (ACTN); protein phosphatase,Drosophila (PTTS); Strongylocentrotus calcium-binding protein (SPEC); Lytechinus purpuratus SPEC resembling protein (LPS); Aequorin and luciferin binding protein (AEQ); calcium vector protein, Branchiostoma (CVP); 1F8 and TB 17 (1F8); calpain and sorcin (CALP); surface protein, Plasmodium (PFS); sarcoplasm calcium-binding protein (SARC); visinin and recoverin (VIS); calcium-binding protein, Saccharopolyspora (CMSE); Tetrahymena calcium-binding protein (TCBP); CAM related gene product, Homo (CRGP); or protein kinase, Plasmodium (PFPK). An EF hand motif useful in the invention also can be a canonical EF hand motif as shown in FIG. 6 or a peptide having significant amino acid homology to a naturally occurring EF hand, for example, at least 60%, 70%, 80%, 90% or 95% amino acid identity with a naturally occurring EF hand such as a member of one of the subfamilies described above. A variety of naturally occurring EF hands are known in the art, as described, for example, in Kawasaki and Kretsinger, Protein Profile 1:343-517 (1994), and Nakayama and Kretsinger, Annu. Rev. Biophys. Biomol. Struct. 23:473-507 (1994). Furthermore, methods of genetically engineering an EF hand motif or the coordination site of an EF hand motif also are well known in the art. See, for example, Vazquez-Ibar et al., Proc. Natl. Acad. Sci. USA 99:3487-3492 (2002).

Lanthanide-binding sites useful in a lanthanide donor complex further include chimeric helix-turn-helix/EF hand peptides, which are helix-turn-helix DNA binding motifs redesigned to include a lanthanide binding site. Such lanthanide-binding sites include, without limitation, the peptide “P3W” (TERRQQLDKDGDGTIDEREIKIWFQNKRAKIK; SEQ ID NO: 101) as described in Welch et al., Proc. Natl. Acad. Sci. USA 100:3725-3730 (2003).

Additional peptide lanthanide-binding sites are known in the art and include, yet are not limited to, those in which the lanthanide-binding site appears to be adventitious or is an intrinsic calcium-binding site. As non-limiting examples, lanthanide ions bind strongly to Bacillus subtilus PyrR (Tomchick et al., Structure 6:337-350 (1998)) and the cadherin NCD1 (Moore et al., J. Am. Chem. Soc. 120:7105-7106 (1998)). See, also, Pidcock and Moore, J. Biol. Inorg. Chem. 6:479-489 (2001). Peptide lanthanide-binding sites also include those identified using screening protocols based, for example, on terbium luminescence (Franz et al., Chem. BioChem. 4:265 (2003); and Nitz et al., Chem. BioChem. 4:272 (2003)) and those identified using similar screening assays.

A lanthanide-binding site useful in a lanthanide donor complex also can be a cryptate, which is a macropolycyclic compound that acts as a cage, trapping a lanthanide ion and protecting it from solvent. The cryptate cage itself acts as an antenna for the trapped lanthanide ion, specifically by absorbing excitation light and transferring the energy to the ion and by protecting it from quenching by water. A variety of lanthanide cryptates are useful in the invention including, but not limited to, trisbipyridine (TBP) lanthanide cryptates and derivatives thereof. Such cryptates, which are tightly associated with their ions, are highly stable in biological media. Lanthanide cryptates useful in the invention include, without limitation, trisbipyridine europium cryptates; trisbipyridine tetracarboxylate (TBP4COOH) europium cryptates; trisbipyridine pentacarboxylate europium cryptates and pyridine bipyridine tetracarboxylate (PBP4COOH) europium cryptates. One skilled in the art understands that cryptate derivatives containing multiple carboxylic groups such as TBP4COOH or PBP4COOH can be significantly more luminescent than their parent cryptate. These and other lanthanide cryptates are well known in the art, as described, for example, in Selvin et al., Ann. Rev. Biomol. Struct. 31:275-302 (2002); Mathis, Clin. Chem. 41:1391-1397 (1995); and Mathis, J. Clin. Ligand Assay 20:141-147 (1997).

Lanthanide-binding sites useful in a lanthanide donor complex further include 2-hydroxyisophthalamide, a molecule which forms luminescent and highly stable complexes with lanthanides such as Sm³⁺, Eu³⁺, Tb³⁺ and Dy³⁺ (Petoud et al., J. Am. Chem. Soc. 125:13324-13325 (2003)). The 2-hydroxyisophthalamide group is a very good ligand for lanthanide ions, providing, for example, excellent sensitization of Tb³⁺ through a particularly efficient ligand-to-lanthanide energy transfer process. The quantum yields of 2-hydroxyisophthalamide lanthanide chelates can be quite high (Φ>0.5), and complexes formed with 2-hydroxyisophthalamides are generally highly soluble and stable in water at physiological pH (Petoud et al., supra, 2003).

A lanthanide-binding site useful in a lanthanide donor complex also can be a β-diketonate such as, without limitation, a Eu³⁺-β-diketonate (2-naphthoyltrifluoroacetonate)-trioctylphosphine oxide ternary fluorescent complex. Such lanthanide-binding sites are well known in the art as described, for example, in Diamandis, Clin. Biochem. 21:139-150 (1988), and are commercially available, for example, as part of the DELFIA® system (Perkin-Elmer).

One skilled in the art understands that these and other lanthanide-binding sites can be useful as part of a lanthanide donor complex in the clostridial toxin substrates and methods of the invention. Such lanthanide-binding sites encompass, but are not limited to, those containing 4,7-bis(chlorosulfodiphenyl)-1,10, phenanthroline-2,9-dicarboxylic acid (“FIAgen” system; Diamandis et al., Anal. Chem. 62:1149A-1157A (1990)) and those containing 5-fluorosalicylate-Tb³⁺-EDTA (“enzyme-amplified time-resolved fluoroimmunoassay” system; Chrisopoulos and Diamandis, Anal. Chem. 64:342-346 (1992)). See, also, Cooper and Sammes, J. Chem. Soc. Perkin Trans. 28:1675-1700; Jones et al., J. Fluoresc. 11:13-21 (2001); and Kolb et al. in Devlin (Ed.), High Throughput Screening: The Discovery of Bioactive Substances pages 345-360 New York: Marcel Dekker (1997)). One skilled in the art understands that these and other peptide, peptidomimetic and small molecule lanthanide-binding sites can be incorporated into a lanthanide donor complex in a substrate of the invention.

Lanthanide-binding sites useful in a lanthanide donor complex further include, without limitation, those with an affinity for a lanthanide ion in the nanomolar to picomolar range. In particular embodiments, a lanthanide-binding site useful in the invention has Kd for a lanthanide ion of less than 10 μM, less than 5 μM, less than 1 μM, less than 500 nM, less than 250 nM, less than 100 nM, less than 50 nM, less than 10 nM, less than 1 nM or less than 0.1 nM. In further embodiments, a lanthanide-binding site useful in the invention has Kd for a lanthanide ion of less than 100 nM, less than 90 nM, less than 80 nM, less than 70 nM, less than 60 nM, less than 50 nM, less than 40 nM, less than 30 nM, less than 20 nM, or less than 10 nM. In still further embodiments, a lanthanide-binding site useful in the invention has Kd for a lanthanide ion of less than 1×10⁻⁹ M, less than 1×10⁻¹⁰ M, less than 1×10⁻¹¹ M, less than 1×10⁻¹² M, less than 1×10⁻¹³ M, less than 1×10⁻¹⁴ M, less than 1×10⁻¹⁵ M, less than 1×10⁻¹⁶ M, less than 1×10⁻¹⁷ M, less than 1×10⁻¹⁸ M, less than 1×10⁻¹⁹ M or less than 1×10⁻²⁰ M.

As used herein, the term “acceptor” means a molecule that can absorb energy from, and upon excitation of, a lanthanide donor complex. An acceptor useful in a clostridial toxin substrate has an absorbance spectrum which overlaps the emission spectrum of the lanthanide donor complex included in the substrate. An acceptor useful in the invention generally has rather low absorption at a wavelength suitable for excitation of the antenna incorporated in the lanthanide donor complex.

As set forth above, an acceptor has an absorbance spectrum that overlaps the emission spectrum of the lanthanide donor complex. The term “overlapping,” as used herein in reference to the absorbance spectrum of an acceptor and the emission spectrum of a lanthanide donor complex, means an absorbance spectrum and emission spectrum that are partly or entirely shared. Thus, in such overlapping spectra, the high end of the range of the emission spectrum of the lanthanide donor complex is higher than the low end of the range of the absorbance spectrum of the acceptor.

A clostridial toxin substrate useful in the invention contains a cleavage site that “intervenes” between a lanthanide donor complex and an acceptor. Thus, the cleavage site is positioned in between the lanthanide donor complex and the acceptor such that proteolysis at the cleavage site results in a first cleavage product containing the lanthanide donor complex and a second cleavage product containing the acceptor. It is understood that all or only a portion of the clostridial toxin recognition sequence may intervene between the lanthanide donor complex and the acceptor.

A clostridial toxin substrate useful in the invention also contains a clostridial toxin recognition sequence which includes a cleavage site. By definition, a clostridial toxin substrate is susceptible to cleavage by at least one clostridial toxin under conditions suitable for clostridial toxin protease activity.

As used herein, the term “clostridial toxin recognition sequence” means a scissile bond together with adjacent or non-adjacent recognition elements, or both, sufficient for detectable proteolysis at the scissile bond by a clostridial toxin under conditions suitable for clostridial toxin protease activity. A variety of clostridial toxin recognition sequences are discussed hereinbelow.

In particular embodiments, a clostridial toxin substrate useful in the invention is a peptide or peptidomimetic having a defined length. A clostridial toxin substrate can be, for example, a peptide or peptidomimetic having at least 100, at least 150, at least 200, at least 250, at least 300, at least 350 or at least 500 residues. In other embodiments, a clostridial toxin substrate has at most 20 residues, at most 30 residues, at most 40 residues, at most 50 residues, at most 100 residues, at most 150 residues, at most 200 residues, at most 250 residues, at most 300 residues, at most 350 residues or at most 400 residues.

It is understood that a clostridial toxin substrate useful in the invention optionally can include one or more additional components. As a non-limiting example, a flexible spacer sequence such as GGGGS (SEQ ID NO: 21) can be included in a clostridial toxin substrate useful in the invention. A useful clostridial toxin substrate further can include, without limitation, one or more of the following: an affinity tag such as HIS6; biotin or a biotinylation sequence; an epitope such as FLAG, hemagluttinin (HA), c-myc, or AU1; an immunoglobulin hinge region; an N-hydroxysuccinimide linker; a peptide or peptidomimetic hairpin turn; or a hydrophilic sequence or another component or sequence that, for example, facilitates purification or promotes the solubility or stability of the clostridial toxin substrate.

A clostridial toxin substrate of the invention contains a lanthanide donor complex and an acceptor, where the clostridial toxin cleavage site is positioned between the lanthanide donor complex and acceptor. In one embodiment, the acceptor is positioned carboxy-terminal of the cleavage site while the lanthanide donor complex is positioned amino-terminal of the cleavage site. In another embodiment, the acceptor is positioned amino-terminal of the cleavage site while the lanthanide donor complex is positioned carboxy-terminal of the cleavage site.

Substrates useful in the invention can be prepared by recombinant methods or using synthetic chemical methods, or a combination thereof. As described herein in Example I, a fusion protein containing GFP fused to a BoNT/A clostridial toxin recognition sequence and a carboxy-terminal cysteine was prepared by recombinant methods. The carboxy-terminal cysteine was used for attachment of a lanthanide donor complex to produce the complete clostridial toxin substrate. Recombinant methods for preparation of clostridial toxin substrates which are fusion proteins are well known in the art as described, for example, in Ausubel, Current Protocols in Molecular Biology John Wiley & Sons, Inc., New York 2000.

Routine chemical methods suitable for modifying a protein, peptide or peptidomimetic to contain a lanthanide donor complex or acceptor or both are well known in the art (Fairclough and Cantor, Methods Enzymol. 48:347-379 (1978); Glaser et al., Chemical Modification of Proteins Elsevier Biochemical Press, Amsterdam (1975); Haugland, Excited States of Biopolymers (Steiner Ed.) pp. 29-58, Plenum Press, New York (1983); Means and Feeney, Bioconjugate Chem. 1:2-12 (1990); Matthews et al., Methods Enzymol. 208:468-496 (1991); Lundblad, Chemical Reagents for Protein Modification 2nd Ed., CRC Press, Boca Ratan, Fla. (1991); Haugland, supra, 1996). As non-limiting examples, a lanthanide donor complex can include an amine-reactive group such as an isothiocyanate (Li and Selvin, Bioconj. Chem. 8:127-132 (1997) or a thiol-reactive group such as a maleimide, bromoacetamide or pyridyl dithio (Chen and Selvin, Bioconjug. Chem. 10:311-315 (1999)). A thiol-reactive lanthanide donor complex is conveniently attached, for example, to a cysteine residue in the substrate. Where a portion of the clostridial toxin substrate is prepared using recombinant techniques, it is understood that a cysteine residue can be engineered at the appropriate position of the substrate for attachment of the lanthanide donor complex (see Example I). Haloacetyl labeling reagents also can be used to couple a lanthanide donor complex or acceptor in preparing a clostridial toxin substrate useful in the invention. See, for example, Wu and Brand, supra, 1994.

Cross-linker moieties also can be useful for preparing a clostridial toxin substrate of the invention. Cross-linkers are well known in the art and include homo- and hetero-bifunctional cross-linkers such as BMH and SPDP. Where the lanthanide-binding site or acceptor is a protein, well known chemical methods for specifically linking molecules to the amino- or carboxy-terminus of a protein can be employed. See, for example, “Chemical Approaches to Protein Engineering” in Protein Engineering: A Practical Approach Rees et al. (Eds) Oxford University Press, 1992.

Lanthanide atoms and DTPA and TTHA chelates are available from a variety of commercial sources including Invitrogen and Sigma. Furthermore, synthesis and purification of DTPA-CS124 and TTHA-CS124 can be routinely performed, for example, as described in Li and Selvin, J. Am. Chem. Soc. 117:8132 (1995). Trisbipyridine (TBP) and tetracarboxylate (TBP4COOH) europium cryptates are commercially available, for example, from CIS Bio International (Bedford, Mass.) or can be prepared by routine methods. One skilled in the art understands that these and other routine recombinant and synthetic chemical methods can be used to prepare a clostridial toxin substrate useful in the invention.

Further provided herein is a method of determining the presence or activity of a clostridial toxin by (a) treating with a sample, under conditions suitable for clostridial toxin protease activity, a clostridial toxin substrate containing (i) a lanthanide donor complex; (ii) an acceptor having an absorbance spectrum overlapping the emission spectrum of the lanthanide donor complex; and (iii) a clostridial toxin recognition sequence containing a cleavage site that intervenes between the lanthanide donor complex and the acceptor, where, under the appropriate conditions, resonance energy transfer is exhibited between the lanthanide donor complex and the acceptor; (b) exciting an antenna of said lanthanide donor complex; and (c) determining resonance energy transfer of the treated substrate relative to a control substrate, where a difference in resonance energy transfer of the treated substrate as compared to the control substrate is indicative of the presence or activity of the clostridial toxin. In one embodiment, a method of the invention is practiced with a clostridial toxin substrate which includes a lanthanide donor complex having a fluorescence lifetime of at least 500 μs. In another embodiment, a method of the invention is practiced with a clostridial toxin substrate which includes a lanthanide donor complex having a fluorescence quantum yield of at least 0.05. In still another embodiment, a method of the invention is practiced with a clostridial toxin substrate which includes a lanthanide donor complex having a fluorescence quantum yield of at least 0.5.

A lanthanide donor complex includes a lanthanide ion such as, without limitation, a terbium ion, europium ion, samarium ion or dysprosium ion. Lanthanide-binding sites useful a lanthanide donor complex encompass, but are not limited to, those having an affinity for a lanthanide ion of at least 5 μM, including, without limitation, peptides and peptidomimetics such as those including the coordination site of an EF hand motif or including an EF hand motif. A lanthanide-binding site useful in a lanthanide donor complex can be, without limitation, a thiol-reactive chelator; diethylenetriaminepentacetic acid (DTPA); β-diketone chelate; polyaminopolycarboxylic acid chelate; calixarene chelate; polyphenol; DOTA; pyridine; polypyridine; trisbipyridine (TBP) cryptate; trisbipyridine tetracarboxylate (TBP4COOH) cryptate; trisbipyridine pentacarboxylate (TBP5COOH) cryptate; or pyridine bipyridine tetracarboxylate (PBP4COOH).

In a method of the invention, the lanthanide donor complex includes an antenna, which can be separate from, or incorporated within, the lanthanide-binding site. Thus, a method of the invention can be practiced, for example, with an antenna which is carbostyryl124 (CS124), tryptophan, or 2-hydroxyisophthalamide. In one embodiment, a method of the invention is practiced with a clostridial toxin substrate in which the lanthanide donor complex includes carbostyryl124 as the antenna. In another embodiment, a method of the invention is practiced with a clostridial toxin substrate in which the lanthanide donor complex is CS124-DTPA-EMCH—Tb.

A method of the invention can be practiced with a clostridial toxin substrate which incorporates any of a variety of acceptors including, without limitation, acceptor fluorophores such as green fluorescent protein (GFP), blue fluorescent protein (BFP), yellow fluorescent protein (YFP), cyan fluorescent protein (CFP) and red fluorescent protein (RFP). In one embodiment, a method of the invention is practiced with a clostridial toxin substrate which includes green fluorescent protein as the acceptor. Non-fluorescent acceptors such as heme proteins also are useful in the methods of the invention.

It is understood that a method of the invention can be practiced using a clostridial toxin substrate which includes any of a variety of recognition sequences. In one embodiment, the recognition sequence is a BoNT/A recognition sequence such as, without limitation, a BoNT/A recognition sequence containing at least six consecutive residues of SNAP-25, where the six consecutive residues include Gln-Arg, or a peptidomimetic thereof. Such a BoNT/A recognition sequence can include, for example, residues 134 to 206 of SEQ ID NO: 2. A recognition sequence useful in a method of the invention also can be, without limitation, a BoNT/B recognition sequence. Such a BoNT/B recognition sequence can contain, for example, at least six consecutive residues of VAMP, where the six consecutive residues include Gln-Phe, or a peptidomimetic thereof. In a further embodiment, a method of the invention is practiced with a clostridial toxin substrate which includes a BoNT/C1 recognition sequence. Such a BoNT/C1 recognition sequence can contain, without limitation, at least six consecutive residues of syntaxin, where the six consecutive residues include Lys-Ala, or a peptidomimetic thereof. A BoNT/C1 recognition sequence useful in the invention also can contain at least six consecutive residues of SNAP-25, where the six consecutive residues include Arg-Ala, or a peptidomimetic thereof.

In a further embodiment, a method of the invention is practiced with a clostridial toxin substrate which includes a BoNT/D recognition sequence. Such a BoNT/D recognition sequence can contain, for example, at least six consecutive residues of VAMP, where the six consecutive residues include Lys-Leu, or a peptidomimetic thereof. A recognition sequence useful in the invention also can be, for example, a BoNT/E recognition sequence. Such a BoNT/E recognition sequence can contain, without limitation, at least six consecutive residues of SNAP-25, where the six consecutive residues include Arg-Ile, or a peptidomimetic thereof. In yet another embodiment, a method of the invention is practiced with a clostridial toxin substrate which includes a BoNT/F recognition sequence. BoNT/F recognition sequences useful in the invention encompass, without limitation, those having at least six consecutive residues of VAMP, where the six consecutive residues include Gln-Lys, or a peptidomimetic thereof. A method of the invention additionally can be practiced with a clostridial toxin substrate which includes a BoNT/G recognition sequence. Such BoNT/G recognition sequences encompass, without limitation, those having at least six consecutive residues of VAMP, where the six consecutive residues include Ala-Ala, or a peptidomimetic thereof. In still a further embodiment, a recognition sequence useful in the invention is a TeNT recognition sequence. Such a TeNT recognition sequence can be, without limitation, a sequence containing at least six consecutive residues of VAMP, where the six consecutive residues include Gln-Phe, or a peptidomimetic thereof.

In particular embodiments, a method of the invention is practiced with a clostridial toxin substrate, such as one including a lanthanide donor complex in which the lanthanide ion is a terbium ion or one in which the lanthanide-binding site includes the coordination site of an EF hand motif, which is a peptide or peptidomimetic having at most 300 residues. In a further embodiment, a method of the invention is practiced with a clostridial toxin substrate which is a peptide or peptidomimetic having at most 150 residues. In a method of the invention, a clostridial toxin substrate of the invention can be cleaved with a range of activities. In one embodiment, a method of the invention is practiced under conditions such that the clostridial toxin substrate is cleaved with an activity of at least 1 nanomole/minute/milligram toxin. In another embodiment, a method of the invention is practiced under conditions such that the clostridial toxin substrate is cleaved with an activity of at least 20 nanomoles/minute/milligram toxin. In a further embodiment, a method of the invention is practiced under conditions such that the clostridial toxin substrate is cleaved with an activity of at least 100 nanomoles/minute/milligram toxin. The methods of the invention can be useful for determining the presence or activity of a clostridial toxin in any of a variety of samples including, but not limited to, crude cell lysates; isolated clostridial toxins such as isolated clostridial toxin light chains; and formulated clostridial toxin products including, but not limited to, formulated BoNT/A, BoNT/B and BoNT/E toxin products.

As discussed further below, it is understood that the methods of the invention are applicable to crude samples as well as highly purified dichain and single chain toxins. As non-limiting examples, a method of the invention can be useful to determine the presence or activity of a clostridial toxin in a food or beverage sample; to assay a sample from a human or animal, for example, exposed to a clostridial toxin or having one or more symptoms of a clostridial toxin; to follow activity during production and purification of clostridial toxin; or to assay formulated clostridial toxin products such as pharmaceuticals or cosmetics.

A variety of samples are useful in the methods of the invention. As used herein, the term “sample” means any biological matter that contains or potentially contains an active clostridial toxin. Thus, the term sample encompasses, but is not limited to, purified or partially purified clostridial toxin; recombinant single chain or dichain toxin with a naturally or non-naturally occurring sequence; recombinant clostridial toxin with a modified protease specificity; recombinant clostridial toxin with an altered cell specificity; chimeric toxin containing structural elements from multiple clostridial toxin species or subtypes; bulk toxin; formulated toxin product; cells or crude, fractionated or partially purified cell lysates, for example, engineered to include a recombinant nucleic acid encoding a clostridial toxin; bacterial, baculoviral and yeast lysates; raw, cooked, partially cooked or processed foods; beverages; animal feed; soil samples; water samples; pond sediments; lotions; cosmetics; and clinical formulations. It further is understood that the term sample encompasses tissue samples, including, without limitation, mammalian tissue samples, livestock tissue samples such as sheep, cow and pig tissue samples; primate tissue samples; and human tissue samples. Such samples encompass, without limitation, intestinal samples such as infant intestinal samples, and tissue samples obtained from a wound.

As discussed further below, a variety of conditions suitable for clostridial toxin protease activity are useful in the methods of the invention. For example, conditions suitable for clostridial toxin protease activity can be provided such that at least 10% of the substrate is cleaved. Similarly, conditions suitable for clostridial toxin protease activity can be provided such that at least 20%, 30%, 40%, 50%, 60%, 70%, 80%, 90% or 95% of the clostridial toxin substrate is cleaved, or such that 100% of the clostridial toxin substrate is cleaved. In one embodiment, the conditions suitable for clostridial toxin protease activity are provided such that the assay is linear. In another embodiment, conditions suitable for clostridial toxin protease activity are provided such that at least 90% of the clostridial toxin substrate is cleaved. In a further embodiment, conditions suitable for clostridial toxin protease activity are provided such that at most 25% of the clostridial toxin substrate is cleaved. In yet further embodiments, conditions suitable for clostridial toxin protease activity are provided such that at most 5%, 10%, 15% or 20% of the clostridial toxin substrate is cleaved.

In the methods of the invention, the clostridial toxin substrate can be treated with a sample in solution phase. As used herein in reference to a clostridial toxin substrate, the term “in solution phase” means that the substrate is soluble and, during proteolysis, is not constrained or immobilized on a solid support such as a bead, column or dish.

In the methods of the invention, a sample is treated with a clostridial toxin substrate under conditions suitable for clostridial toxin protease activity. Exemplary conditions suitable for clostridial toxin protease activity are well known in the art, and further can be determined by routine methods. See, for example, Hallis et al., J. Clin. Microbiol. 34:1934-1938 (1996); Ekong et al., Microbiol. 143:3337-3347 (1997); Shone et al., WO 95/33850; Schmidt and Bostian, supra, 1995; Schmidt and Bostian, supra, 1997; Schmidt et al., supra, 1998; and Schmidt and Bostian, U.S. Pat. No. 5,965,699. It is understood that conditions suitable for clostridial toxin protease activity can depend, in part, on the specific clostridial toxin type or subtype being assayed and the purity of the toxin preparation. Conditions suitable for clostridial toxin protease activity generally include a buffer, such as HEPES, Tris or sodium phosphate, typically in the range of pH 5.5 to 9.5, for example, in the range of pH 6.0 to 9.0, pH 6.5 to 8.5 or pH 7.0 to 8.0. Conditions suitable for clostridial toxin protease activity also can include, if desired, dithiothreitol, β-mercaptoethanol or another reducing agent, for example, where a dichain toxin is being assayed (Ekong et al., supra, 1997). In one embodiment, the conditions include DTT in the range of 0.01 mM to 50 mM; in other embodiments, the conditions include DTT in the range of 0.1 mM to 20 mM, 1 to 20 mM, or 5 to 10 mM. If desired, an isolated clostridial toxin or sample can be pre-incubated with a reducing agent, for example, with 10 mM dithiothreitol (DTT) for about 30 minutes prior to addition of clostridial toxin substrate.

Clostridial toxins are zinc metalloproteases, and a source of zinc, such as zinc chloride or zinc acetate, typically in the range of 1 to 500 μM, for example, 5 to 10 μM can be included, if desired, as part of the conditions suitable for clostridial toxin protease activity. One skilled in the art understands that zinc chelators such as EDTA generally are excluded from a buffer for determining the presence or activity of a clostridial toxin.

Conditions suitable for clostridial toxin protease activity can optionally include a detergent such as TWEEN-20, which can be used, for example, in place of bovine serum albumin. TWEEN-20 can be provided, for example, in the range of 0.001% to 10% (v/v), or in the range of 0.01% to 1.0% (v/v). As a non-limiting example, TWEEN-20 can be included at a concentration of 0.1 % (v/v).

Conditions suitable for clostridial toxin protease activity also can include, if desired, bovine serum albumin (BSA) or another agent which acts as a protein stabilizer, solubilizing agent or blocker of surface loss. As an example, when included, BSA typically is provided in the range of 0.1 mg/ml to 10 mg/ml. In one embodiment, BSA is included at a concentration of 1 mg/ml. See, for example, Schmidt and Bostian, supra, 1997. In another embodiment, BSA is included at a concentration of 0.1 % (w/v).

The amount of clostridial toxin substrate can be varied in a method of the invention. A clostridial toxin substrate can be supplied, for example, at a concentration of 1 μM to 500 μM, 1 μM to 50 μM, 1 μM to 30 μM, 5 μM to 20 μM, 50 μM to 3.0 mM, 0.5 mM to 3.0 mM, 0.5 mM to 2.0 mM, or 0.5 mM to 1.0 mM. The skilled artisan understands that the concentration of clostridial toxin substrate or the amount of sample can be limited, if desired, such that the assay is linear. In one embodiment, a method of the invention relies on a clostridial toxin substrate concentration of less than 100 μM. In further embodiments, a method of the invention relies on a clostridial toxin substrate concentration of less than 50 μM or less than 25 μM. In a further embodiment, a method of the invention relies on a clostridial toxin substrate concentration of 10 μM to 20 μM. If desired, a linear assay also can be performed by mixing clostridial toxin substrate with corresponding, “unlabeled” substrate which lacks a functional lanthanide donor complex. The appropriate dilution can be determined, for example, by preparing serial dilutions of clostridial toxin substrate in the corresponding unlabeled substrate.

The concentration of purified or partially purified clostridial toxin to be assayed in a method of the invention generally is in the range of about 0.0001 ng/ml to 500 μg/ml toxin, for example, about 0.0001 ng/ml to 50 μg/ml toxin, 0.001 ng/ml to 500 μg/ml toxin, 0.001 ng/ml to 50 μg/ml toxin, 0.0001 to 5000 ng/ml toxin, 0.001 ng/ml to 5000 ng/ml, 0.01 ng/ml to 5000 ng/ml, 0.1 ng/ml to 5000 ng/ml, 1 ng/ml to 5000 ng/ml, 10 ng/ml to 5000 ng/ml, 50 ng/ml to 5000 ng/ml, 50 ng/ml to 500 ng/ml or 100 ng/ml to 5000 ng/ml toxin, which can be, for example, purified recombinant dichain or single chain toxin or formulated clostridial toxin product containing human serum albumin and excipients. Generally, the amount of purified toxin assayed in a method of the invention is in the range of 0.1 pg to 100 μg, for example, 0.1 pg to 50 μg or 0.1 pg to 10 μg.

The concentration of purified or partially purified clostridial toxin assayed in a method of the invention can be, for example, in the range of about 0.1 pM to 100 μM, 0.1 pM to 10 μM, 0.1 pM to 1 μM, 0.1 pM to 500 nM, 0.1 pM to 100 nM, for example, 1 pM to 2000 pM, 1 pM to 200 pM, 1 pM to 50 pM, 1 nM to 1 μM, 1 nM to 500 nM, 1 nM to 200 nM, 1 nM to 100 nM, or 3 nM to 100 nM toxin, which can be, for example, purified native or recombinant light chain or dichain toxin or formulated clostridial toxin product containing human serum albumin and excipients. In particular embodiments, the concentration of purified or partially purified recombinant BoNT/A, BoNT/B or BoNT/E light chain or dichain or formulated toxin product is in the range of 1 pM to 2000 pM, 10 pM to 2000 pM, 20 pM to 2000 pM, 40 pM to 2000 pM, or 1 pM to 200 pM. In further embodiments, the concentration of purified or partially purified recombinant BoNT/C light chain or dichain or formulated toxin product is in the range of 1 nM to 200 nM, 4 nM to 100 nM, 10 nM to 100 nM or 4 nM to 60 nM. One skilled in the art understands that the concentration of purified or partially purified clostridial toxin will depend on the serotype of the toxin assayed, as well as the purity or recombinant sequence of the toxin, the presence of inhibitory components, and the assay conditions. It is additionally understood that purified, partially purified or crude samples can be diluted to within a convenient range for assaying for clostridial toxin protease activity against a standard curve. Similarly, it is understood that a sample can be diluted, if desired, such that the assay is linear.

Conditions suitable for clostridial toxin protease activity also generally include, for example, temperatures in the range of about 20° C. to about 45° C., for example, in the range of 25° C. to 40° C., or the range of 35° C. to 39° C. Assay volumes often are in the range of about 5 μl to about 200 μl, for example, in the range of about 10 μl to 100 μl or about 0.5 μl to 100 μl, although nanoliter reaction volumes also can be used with the methods of the invention. Assay volumes also can be, for example, in the range of 100 μl to 2.0 ml or in the range of 0.5 ml to 1.0 ml.

Assay times can be varied as appropriate by the skilled artisan and generally depend, in part, on the concentration, purity and activity of the clostridial toxin. Assay times generally vary, without limitation, in the range of about 15 minutes to about 5 hours. As non-limiting examples, exemplary assay times include incubation, for example, at 37° C. for 30 minutes, 45 minutes, 60 minutes, 75 minutes or 90 minutes. In particular embodiments, at least 10%, 20%, 30%,40%, 50%, 60%, 70%, 80%, 90%, 95% or 100% of the clostridial toxin substrate is cleaved. In further embodiments, the protease reaction is stopped before more than 5%, 10%, 15%, 20%, 25% or 50% of the clostridial toxin substrate is cleaved. It is understood that protease reactions can be terminated by the appropriate reagent, which generally depends on the lanthanide donor complex and other components of the substrate. As a non-limiting example, a protease reaction based on a substrate containing GFP as the fluorescent acceptor can be terminated by the addition of guanidinium chloride, for example, to a final concentration of 1 to 2 M. Protease reactions also can be terminated by addition of H₂SO₄; addition of about 0.5 to 1.0 sodium borate, pH 9.0 to 9.5; or addition of zinc chelators. One skilled in the art understands that protease reactions can be terminated, if desired, prior to exciting the antenna.

As a non-limiting example, conditions suitable for clostridial toxin protease activity such as BoNT/A protease activity can be incubation at 37° C. for 90 minutes in a buffer containing 50 mM HEPES (pH 7.2), 10 μM ZnCl₂, 10 mM DTT, and 0.1% (v/v) TWEEN-20 with 10-16 μM substrate. If desired, samples containing BoNT/A, particularly dichain BoNT/A, can be preincubated with dithiothreitol, for example, for 20 or 30 minutes before addition of substrate. As a further non-limiting example, conditions suitable for BoNT/A protease activity can be incubation at 37° C. in a buffer such as 30 mM HEPES (pH 7.3) containing a reducing agent such as 5 mM dithiothreitol; and a source of zinc such as 25 μM zinc chloride (approximately 7 nM; Schmidt and Bostian, supra, 1997). BSA in the range of 0.1 mg/ml to 10 mg/ml, for example, 1 mg/ml BSA, also can be included when a sample is treated with a clostridial toxin substrate (Schmidt and Bostian, supra, 1997). As another non-limiting example, conditions suitable for clostridial toxin protease activity, for example BoNT/B activity, can be incubation in 50 mM HEPES, pH 7.4, with 10 μM zinc chloride, 1% fetal bovine serum and 10 mM dithiothreitol, with incubation for 90 minutes at 37° C. (Shone and Roberts, Eur. J. Biochem. 225:263-270 (1994); Hallis et al., supra, 1996); or can be, for example, incubation in 40 mM sodium phosphate, pH 7.4, with 10 mM dithiothreitol, optionally including 0.2% (v/v) Triton X-100, with incubation for 2 hours at 37° C. (Shone et al., supra, 1993). Conditions suitable for tetanus toxin protease activity or other clostridial toxin protease activity can be, for example, incubation in 20 mM HEPES, pH 7.2, and 100 mM NaCl for 2 hours at 37° C. with 25 μM peptide substrate (Comille et al., supra, 1994).

The present invention relies, in part, on luminescence resonance energy transfer (LRET), in which a lanthanide ion such as Tb³⁺ or Eu³⁺ transfers energy non-radiatively to an organic acceptor, which may be a fluorophore, through intramolecular long-range dipole-dipole coupling. FRET is dependent on the inverse sixth power of the intramolecular separation of the lanthanide donor complex and acceptor, and for effective transfer, the lanthanide donor complex and acceptor are in close proximity, separated, for example, by about 10 Å to about 100 Å. Effective energy transfer is dependent on the spectral characteristics of the lanthanide donor complex and acceptor as well as their relative orientation (see Clegg, Current Opinion in Biotech. 6:103-110 (1995); and Selvin, Nature Structural Biol. 7:730-734 (2000)).

In a clostridial toxin substrate of the invention, a lanthanide ion and acceptor are selected so that the lanthanide donor complex and acceptor exhibit resonance energy transfer when the antenna of the lanthanide donor complex is excited. As is well known in the art, the efficiency of resonance energy transfer is dependent on the separation distance of the lanthanide ion or other component of the lanthanide donor complex and acceptor as described by the Förster equation, as well as the fluorescent quantum yield of the lanthanide ion and the energetic overlap with the acceptor. In one embodiment, the invention provides a clostridial toxin substrate in which, under optimal conditions, the efficiency of LRET between the lanthanide donor complex and acceptor is at least 10%. In another embodiment, the invention provides a clostridial toxin substrate in which, under optimal conditions, the efficiency of LRET between the lanthanide donor complex and acceptor is at least 20%. In still further embodiments, the invention provides a clostridial toxin substrate in which, under optimal conditions, the efficiency of LRET between the lanthanide donor complex and acceptor is at least 30%, 40%, 50%, 60%, 70% or 80%.

The clostridial toxin substrates of the invention exploit the remarkable luminescent properties of lanthanides, which are their long, millisecond to submillisecond lifetimes, narrow and multiple emission bands in the visible spectrum, and unpolarized emission. Useful lanthanide donor complex/acceptor pairs for use in the clostridial toxin substrates of the invention include, without limitation, CS124-DTPA-EMCH—Tb or another terbium ion complex in combination with a green fluorescent protein or blue fluorescent protein as the acceptor (see Examples I and II). A useful lanthanide donor complex/acceptor pair also can be the lanthanide donor complex Eu-trisbipyridine cryptate (TBP-Eu³⁺, λ_{Ex}337 nm) in combination with the 105 kDa phycobiliprotein acceptor fluorophore, allophycocyanin (Sittampalam et al., Curr. Opin. Chem. Biol. 1:384-391 (1997)). The Eu-trisbipyridine cryptate has two bipyridyl groups that harvest light and channel it to the caged Eu³⁺; Eu³⁺ nonradiatively transfers energy to allophycocyanin when in close proximity to the acceptor, exhibiting greater than 50% transfer efficiency at a lanthanide ion-acceptor distance of 9.5 nm. Furthermore, both TBP-Eu³⁺ and allophycocyanin and their spectroscopic characteristics are very stable in biological media, and allophycocyanin emits (λ_{Em}=665 nm) with the long lifetime of the lanthanide ion, allowing time-resolved detection (Kolb et al., J. Biomol. Screening 1:203-210 (1996)). Methods of preparing substrates containing such donor fluorophore-acceptor pairs are well known in the art as described, for example, in Kolb et al., supra, 1996, and Sittampalam et al., supra, 1997.

A clostridial toxin substrate of the invention contains a clostridial toxin cleavage site which is positioned between a lanthanide donor complex and an acceptor. In one embodiment, the lanthanide donor complex is positioned carboxy-terminal of the cleavage site while the acceptor is positioned amino-terminal of the cleavage site. In another embodiment, the lanthanide donor complex is positioned amino-terminal of the cleavage site while the acceptor is positioned carboxy-terminal of the cleavage site.

One skilled in the art understands that there are several considerations in selecting and positioning a lanthanide donor complex and acceptor in a clostridial toxin substrate of the invention. The lanthanide donor complex and acceptor generally are positioned to minimize interference with substrate binding to, or proteolysis by, the clostridial toxin. Thus, a lanthanide donor complex and acceptor can be selected and positioned, for example, so as to minimize the disruption of bonded and non-bonded interactions that are important for binding, and to minimize steric hindrance. In addition, the spatial distance between the acceptor and lanthanide donor complex generally is limited to achieve efficient energy transfer from the lanthanide donor complex to the acceptor.

As discussed above, efficiency of energy transfer from lanthanide donor complex to acceptor will be dependent, in part, on the spatial separation of the lanthanide donor complex and acceptor. As the distance between the lanthanide donor complex and acceptor increases, there is less energy transfer to the acceptor, and the lanthanide donor complex signal therefore increases, even prior to cleavage. The overall increase in fluorescence yield of the lanthanide donor complex, upon cleavage of the substrate, is dependent upon many factors, including the separation distance between the lanthanide donor complex and acceptor in the substrate, the spectral overlap between the lanthanide donor complex and acceptor, and the concentration of substrate used in an assay. One skilled in the art understands that, as the concentration of substrate increases, intermolecular quenching of the donor, even after proteolytic cleavage, can become a factor. This phenomenon is denoted the “inner filter effect.”

The Föster distance, which is the separation between a donor fluorophore and an acceptor for 50% energy transfer, represents a spatial separation between donor fluorophore and acceptor that provides a good sensitivity. For peptide substrates, adjacent residues are separated by a distance of approximately 3.6 Å in the most extended conformation. However, because peptides and peptidomimetics in solution rarely have a fully extended conformation, a lanthanide donor complex and an acceptor can be more widely separated than expected based on a calculation performed using 3.6 Å per residue and still remain within the Föster distance. In one embodiment, the invention provides a clostridial toxin substrate in which the lanthanide ion or other component of a lanthanide donor complex is spatially separated from an acceptor by a distance of at most 100 Å. In other embodiments, the invention provides a clostridial toxin substrate in which the lanthanide ion or other component of a lanthanide donor complex is spatially separated from an acceptor by a distance of at most 90 Å, 80 Å, 70 Å, 60 Å, 50 Å, 40 Å, 30 Å or 20 Å. In further embodiments, the invention provides a clostridial toxin substrate in which the lanthanide ion or other component of a lanthanide donor complex is spatially separated from an acceptor by a distance of 10 Å to 100 Å, 10 Å to 80 Å, 10 Å to 60 Å, 10 Å to 40 Å, 10 Åto 20 Å, 20 Å to 100 Å, 20Å to 80 Å, 20 Å to 60 Å, 20 Å to 40 Å, 40 Å to 100 Å, 40 Å to 80 Å or 40 Å to 60 Å.

One skilled in the art understands that a clostridial toxin substrate of the invention can be designed, in part, to optimize the efficiency of resonance energy transfer. One skilled in the art understands that lanthanide ions useful in the invention generally have a high quantum yield, and that an acceptor can be selected, if desired, with a high extinction coefficient to maximize the Föster distance. One skilled in the art further understands that fluorescence arising from direct excitation of an acceptor can be difficult to distinguish from fluorescence resulting from resonance energy transfer. Thus, it is recognized that a lanthanide donor complex and acceptor can be selected which have relatively little overlap of their excitation spectra such that the antenna of a lanthanide donor complex can be excited at a wavelength that does not result in direct excitation of the acceptor. It further is recognized that a clostridial toxin substrate of the invention can be designed so that the emission spectra of the lanthanide donor complex and acceptor overlap relatively little such that the two emissions can be readily distinguished. If desired, an acceptor having a high fluorescence quantum yield can be selected.

Proteolysis of a clostridial toxin substrate, and hence the presence or activity of a clostridial toxin, can be detected by a variety of means, for example, by detecting increased luminescence from at least one emission peak of a lanthanide donor complex; by detecting decreased acceptor fluorescence intensity; or by detecting a decreased ratio of fluorescence amplitudes near the acceptor emission maximum to the fluorescence amplitudes near the lanthanide donor complex emission maximum. It is understood that the relevant luminescence intensities are detected at the appropriate selected wavelength or range of wavelengths. Proteolysis of a clostridial toxin substrate, and hence the presence or activity of a clostridial toxin, also can be detected by, for example, a shift in emission maxima from near the acceptor emission maximum to near an emission maximum of the lanthanide ion, or an increased excited state lifetime of the lanthanide ion.

In one embodiment, luminescence intensity of at least one emission peak of the lanthanide donor complex is detected, with increased luminescence intensity indicative of the presence or activity of clostridial toxin. Such increased intensity can be, for example, at least two-fold, three-fold, five-fold, ten-fold, twenty-fold or more relative to luminescence intensity at the same wavelength of the same clostridial toxin substrate not contacted with sample. Such increased intensity also can be, for example, an increase of at least 0.1 -fold, 0.2-fold, 0.3-fold, 0.4-fold, 0.5-fold, 0.6-fold, 0.7-fold, 0.8-fold, 0.9-fold, 1.0-fold, or 1.5-fold relative to luminescence intensity at the same wavelength of the same clostridial toxin substrate not contacted with sample.

For detection of luminescence intensity of a lanthanide donor complex, excitation is set at the wavelength of antenna absorption, and the emission of at least one peak of the lanthanide donor complex is monitored. The emission wavelength of the lanthanide donor complex generally is selected such that little or no contribution from acceptor fluorescence is observed. The presence of acceptor quenches luminescence from the lanthanide donor complex as disclosed herein in Example II. The methods of the invention for determining the presence or activity of a clostridial toxin involve determining resonance energy transfer of a clostridial toxin substrate treated with a sample relative to a control substrate and can be practiced as “fixed-time” assays or as continuous time assays.

In the methods of the invention, luminescence resonance energy transfer of the clostridial toxin treated substrate is determined relative to a control substrate. Such a control substrate can be, without limitation, the same clostridial toxin substrate which is not treated with any sample, or which is treated with a defined sample known to contain one or more clostridial toxins, or with a defined sample known to lack active clostridial toxin. It is clear from the above that a variety of control substrates are useful in the methods of the invention and that a control substrate can be a positive control substrate or a negative control substrate. A control substrate can be, for example, a negative control substrate such as a similar or identical substrate that is contacted with a similar sample that does not contain active clostridial toxin, or that is not contacted with any sample, or which is not susceptible to cleavage by the clostridial toxin. A control substrate also can be, for example, a positive control substrate such as a cleavage product that results from clostridial toxin proteolysis of the clostridial toxin substrate. Such a control substrate can be the lanthanide donor complex-containing cleavage product, the acceptor-containing cleavage product, or a combination of both.

It is understood that the methods of the invention can be automated and can be configured in a high-throughput or ultra high-throughput format using, without limitation, 96-well, 384-well or 1536-well plates. As one example, fluorescence emission can be detected using Molecular Devices FLIPR® instrumentation system (Molecular Devices; Sunnyvale, Calif.), which is designed for 96-well plate assays (Schroeder et al., J. Biomol. Screening 1:75-80 (1996)). FLIPR utilizes a water-cooled 488 nm argon ion laser (5 watt) or a xenon arc lamp and a semiconfocal optical system with a charge-coupled device (CCD) camera to illuminate and image the entire plate. The FPM-2 96-well plate reader (Folley Consulting and Research; Round Lake, Ill.) also can be useful in determining resonance energy transfer in the methods of the invention. One skilled in the art understands that these and other automated systems with the appropriate spectroscopic compatibility such as the ECLIPSE cuvette reader (Varian-Cary; Walnut Creek, Calif.), the SPECTRAₘₐₓ GEMINI XS (Molecular Devices) and other systems from, for example, Perkin Elmer can be useful in the methods of the invention.

Many compounds and proteins present in biological samples are naturally fluorescent; thus, the use of conventional fluorophores can lead to significant limitations in sensitivity. However, non-specific background fluorescence is short-lived, typically having a decay time of only about 10 nanoseconds and therefore dying away much earlier than sample fluorescence. Thus, most background signals can be readily differentiated using time-resolved fluorescence (TRF), which is a quick and convenient assay based on the long-lived fluorescence of the rare earth lanthanides. In time-resolved fluorescence, the detector is gated for a short period of time such that the initial burst of fluorescence, including most of the background fluorescence, is not measured. After the gating period, the longer lasting fluorescence in the sample is measured, substantially enhancing sensitivity. As a non-limiting example, a pulsed excitation source for exciting the antenna of a lanthanide donor complex can be generated using a nitrogen laser (337 nm). Typically, a pulse-width of about 5 nanoseconds is utilized with a 20 to 50-Hz repetition rate. For lifetime measurements, a photomultiplier tube with suitable color filters and counting electronics can be used. For time-delayed spectra, a spectrometer, generally utilizing diffraction gratings, and either a time-gated photomultiplier tube or a CCD, gated electronically or with a mechanical chopper are used. Such instruments are commercially available and are well known in the art as described, for example, in Xiao and Selvin, Rev. Sci. Inst. 70:3877-3881 (1999), Xiao and Selvin, J. Am. Chem. Soc. 123:7067-7073 (2001), and Selvin, supra, 2002.

Specific and distinct cleavage sites for different clostridial toxins are well known in the art. BoNT/A cleaves a Gln-Arg bond; BoNT/B and TeNT cleave a Gln-Phe bond; BoNT/C1 cleaves a Lys-Ala or Arg-Ala bond; BoNT/D cleaves a Lys-Leu bond; BoNT/E cleaves an Arg-Ile bond; BoNT/F cleaves a Gln-Lys bond; and BoNT/G cleaves an Ala-Ala bond (see Table A). In standard nomenclature, the sequence surrounding a clostridial toxin cleavage site is denoted P₅₋P₄₋P₃₋P₂₋P₁₋P₁′-P₂′-P₃′-P₄′-P₅′, with P₁-P₁′ representing the scissile bond. It is understood that a P₁ or P₁′ site, or both, can be substituted with another amino acid or amino acid mimetic in place of the naturally occurring residue. As an example, BoNT/A substrates have been prepared in which the P₁ position (Gln) is modified to be an alanine, 2-aminobutyric acid or asparagine residue; these substrates were hydrolyzed by BoNT/A at the P₁₋Arg bond (Schmidt and Bostian, J. Protein Chem. 16:19-26 (1997)). While it is recognized that substitutions can be introduced at the P₁ position of the scissile bond, for example, a BoNT/A scissile bond, it is further recognized that conservation of the P₁′ residue can be advantageous (Vaidyanathan et al., J. Neurochem. 72:327-337 (1999)). Thus, in particular embodiments, the invention provides a method which relies on a clostridial toxin substrate having a clostridial toxin recognition sequence in which the P₁′ residue is not modified or substituted relative to the naturally occurring residue in a target protein cleaved by the clostridial toxin. In other embodiments, the invention provides a method which relies on a clostridial toxin substrate having a recognition sequence in which the P₁ residue is modified or substituted relative to the naturally occurring residue in a target protein cleaved by the clostridial toxin; such a clostridial toxin substrate retains susceptibility to peptide bond cleavage between the P₁ and P₁′ residues.

TABLE A

BONDS CLEAVED IN HUMAN VAMP-2, SNAP-25
OR SYNTAXIN

| Toxin | Target | P₄-P₃-P₂P₁ - P₁'-P₂'-P₃'P₄' |
|---|---|---|
| BoNT/A | SNAP-25 | Glu-Ala-Asn-Gln—Arg*-Ala-Thr-Lys |
| | | SEQ ID NO: 22 |
| BoNT/B | VAMP-2 | Gly-Ala-Ser-Gln—Phe*-Glu-Thr-Ser |
| | | SEQ ID NO: 23 |
| BoNT/C | syntaxin | Asp-Thr-Lys-Lys—Ala*-Val-Lys-Tyr |
| | | SEQ ID NO: 24 |
| BoNT/D | VAMP-2 | Arg-Asp-Gln-Lys—Leu*-Ser-Glu-Leu |
| | | SEQ ID NO: 25 |
| BoNT/E | SNAP-25 | Gln-Ile-Asp-Arg—Ile*-Met-Glu-Lys |
| | | SEQ ID NO: 26 |
| BoNT/F | VAMP-2 | Glu-Arg-Asp-Gln—Lys*-Leu-Ser-Glu |
| | | SEQ ID NO: 27 |
| BoNT/G | VAMP-2 | Glu-Thr-Ser-Ala—Ala*-Lys-Leu-Lys |
| | | SEQ ID NO: 28 |
| TeNT | VAMP-2 | Gly-Ala-Ser-Gln—Phe*-Glu-Thr-Ser |
| | | SEQ ID NO: 29 |

*Scissile bond shown in bold

SNAP-25, VAMP and syntaxin share a short motif located within regions predicted to adopt an α-helical conformation. This motif is present in SNAP-25, VAMP and syntaxin isoforms expressed in animals sensitive to the neurotoxins. In contrast, Drosophila and yeast homologs that are resistant to these neurotoxins and syntaxin isoforms not involved in exocytosis contain sequence variations in the α-helical motif regions of these VAMP and syntaxin proteins.

Multiple repetitions of the α-helical motif are present in proteins sensitive to cleavage by clostridial toxins: Four copies are naturally present in SNAP-25; two copies are naturally present in VAMP; and two copies are naturally present in syntaxin. Furthermore, peptides corresponding to the specific sequence of the α-helical motifs can inhibit neurotoxin activity in vitro and in vivo, and such peptides can cross-inhibit different neurotoxins. In addition, antibodies raised against such peptides can cross-react among the three target proteins, indicating that this α-helical motif is exposed on the protein surface and adopts a similar configuration in each of the three target proteins. Consistent with these findings, SNAP-25-specific, VAMP-specific and syntaxin-specific neurotoxins cross-inhibit each other by competing for the same binding site, although they do not cleave targets non-specifically. These results indicate that a clostridial toxin recognition sequence can include, if desired, at least one α-helical motif. It is recognized that an α-helical motif is not required for cleavage by a clostridial toxin, as evidenced by 16-mer and 17-mer substrates for BoNT/A known in the art.

Although multiple α-helical motifs are found in the naturally occurring SNAP-25, VAMP and syntaxin target proteins, a clostridial toxin recognition sequence useful in a clostridial toxin substrate can have a single α-helical motif. In particular embodiments, a method of the invention relies on a clostridial toxin recognition sequence including two or more α-helical motifs. A BoNT/A or BoNT/E recognition sequence can include, for example, the S4 α-helical motif, alone or combined with one or more additional α-helical motifs; a BoNT/B, BoNT/G or TeNT recognition sequence can include, for example, the V2 α-helical motif, alone or combined with one or more additional α-helical motifs; a BoNT/C1 recognition sequence can include, for example, the S4 α-helical motif, alone or combined with one or more additional α-helical motifs, or the X2 α-helical motif, alone or combined with one or more additional α-helical motifs; and a BoNT/D or BoNT/F recognition sequence can include, for example, the V1 α-helical motif, alone or combined with one or more additional α-helical motifs.

### BoNT/A Recognition Sequences

As used herein, the term “botulinum toxin serotype A recognition sequence” is synonymous with “BoNT/A recognition sequence” and means a scissile bond together with adjacent or non-adjacent recognition elements, or both, sufficient for detectable proteolysis at the scissile bond by a BoNT/A under conditions suitable for clostridial toxin protease activity. A scissile bond cleaved by BoNT/A can be, for example, Gln-Arg.

A variety of BoNT/A recognition sequences are well known in the art and are useful in the invention. A BoNT/A recognition sequence can have, for example, residues 134 to 206 or residues 137 to 206 of human SNAP-25 (Ekong et al., supra, 1997; U.S. Pat. No. 5,962,637). A BoNT/A recognition sequence also can include, without limitation, the sequence Thr-Arg-Ile-Asp-Glu-Ala-Asn-Gln-Arg-Ala-Thr-Lys-Met (SEQ ID NO: 30) or a peptidomimetic thereof, which corresponds to residues 190 to 202 of human SNAP-25; Ser-Asn-Lys-Thr-Arg-Ile-Asp-Glu-Ala-Asn-Gln-Arg-Ala-Thr-Lys (SEQ ID NO: 31) or a peptidomimetic thereof, which corresponds to residues 187 to 201 of human SNAP-25; Ser-Asn-Lys-Thr-Arg-Ile-Asp-Glu-Ala-Asn-Gln-Arg-Ala-Thr-Lys-Met (SEQ ID NO: 32) or a peptidomimetic thereof, which corresponds to residues 187 to 202 of human SNAP-25; Ser-Asn-Lys-Thr-Arg-Ile-Asp-Glu-Ala-Asn-Gln-Arg-Ala-Thr-Lys-Met-Leu (SEQ ID NO: 33) or a peptidomimetic thereof, which corresponds to residues 187 to 203 of human SNAP-25; Asp-Ser-Asn-Lys-Thr-Arg-Ile-Asp-Glu-Ala-Asn-Gln-Arg-Ala-Thr-Lys-Met (SEQ ID NO: 34) or a peptidomimetic thereof, which corresponds to residues 186 to 202 of human SNAP-25; or Asp-Ser-Asn-Lys-Thr-Arg-Ile-Asp-Glu-Ala-Asn-Gln-Arg-Ala-Thr-Lys-Met-Leu (SEQ ID NO: 35) or a peptidomimetic thereof, which corresponds to residues 186 to 203 of human SNAP-25. See, for example, Schmidt and Bostian, J. Protein Chem. 14:703-708 (1995); Schmidt and Bostian, supra, 1997; Schmidt et al., FEBS Letters 435:61-64 (1998); and Schmidt and Bostian, U.S. Pat. No. 5,965,699). If desired, a similar BoNT/A recognition sequence can be prepared from a corresponding (homologous) segment of another BoNT/A-sensitive SNAP-25 isoform or homolog such as, for example, murine, rat, goldfish or zebrafish SNAP-25 or can be any of the peptides described herein or known in the art, for example, in U.S. Pat. No. 5,965,699.

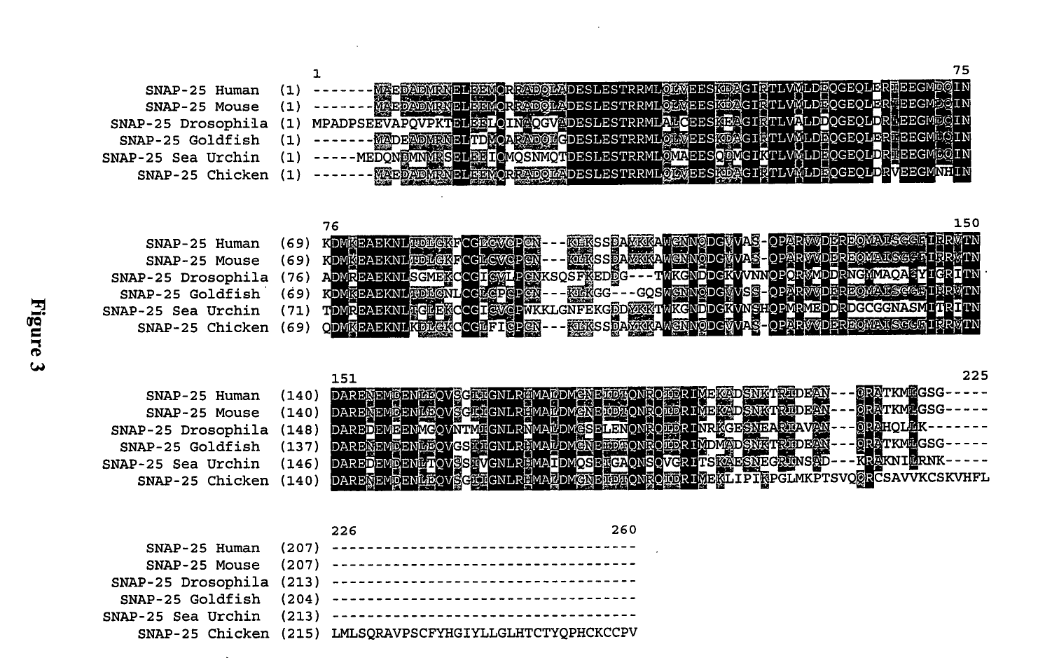

A BoNT/A recognition sequence useful in the invention can correspond to a segment of a protein that is sensitive to cleavage by botulinum toxin serotype A, or can be substantially similar to a segment of a BoNT/A-sensitive protein. As illustrated in Table B, a variety of naturally occurring proteins sensitive to cleavage by BoNT/A are known in the art and include, for example, human, mouse and rat SNAP-25; and goldfish SNAP-25A and SNAP-25B. Thus, a BoNT/A recognition sequence useful in the invention can correspond, for example, to a segment of human SNAP-25, mouse SNAP-25, rat SNAP-25, goldfish SNAP-25A or 25B, or another naturally occurring protein sensitive to cleavage by BoNT/A. Furthermore, comparison of native SNAP-25 amino acid sequences cleaved by BoNT/A reveals that such sequences are not absolutely conserved (see Table B and FIG. 3), indicating that a variety of amino acid substitutions and modifications relative to a naturally occurring BoNT/A-sensitive SNAP-25 sequence can be tolerated in a BoNT/A recognition sequence useful in the invention.

TABLE B
Cleavage of SNAP-25 and related proteins^{a,b,c,d}

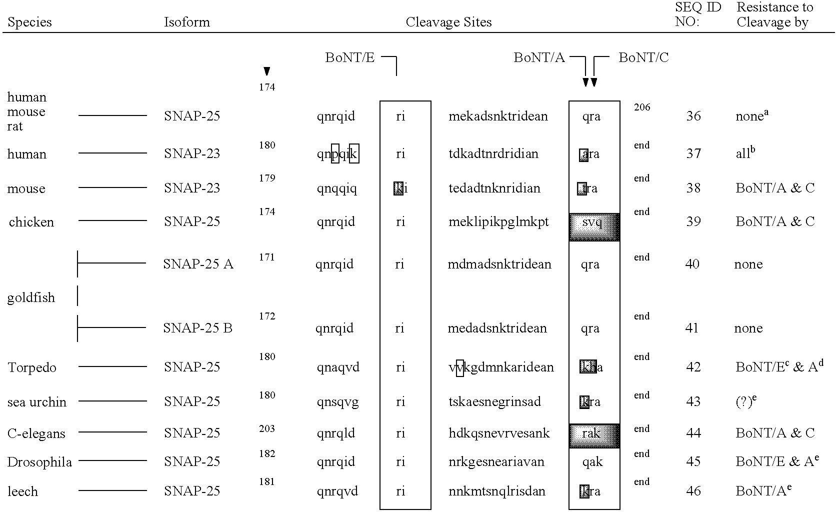

^{a}= In vitro cleavage of SNAP-25 requires 1000-fold higher BoNT/C concentration than BoNT/A or /E.
^{b}= Substitution ofp182r, or k185dd (boxes) induces susceptibility toward BoNT/E.
^{c}= Resistance to BoNT/A possibly due to d189 or e189 substitution by v189, see box.
^{d}= Note that Torpedo is susceptible to BoNT/A.
^{e}= Note the presence of several non-conservative mutations around putative cleavage sites.

A clostridial toxin substrate, such as a substrate containing a BoNT/A recognition sequence, can have one or multiple modifications as compared to a naturally occurring sequence that is cleaved by the corresponding clostridial toxin. As an example, as compared to a 17-mer corresponding to residues 187 to 203 of human SNAP-25, substitution of Asp193 with Asn in the BoNT/A substrate resulted in a relative rate of proteolysis of 0.23; substitution of Glu194 with Gln resulted in a relative rate of 2.08; substitution of Ala195 with 2-aninobutyric acid resulted in a relative rate of 0.38; and substitution of Gln197 with Asn, 2-aminobutyric acid or Ala resulted in a relative rate of 0.66, 0.25, or 0.19, respectively (see Table C). Furthermore, substitution of Ala199 with 2-aninobutyric acid resulted in a relative rate of 0.79; substitution of Thr200 with Ser or 2-aminobutyric acid resulted in a relative rate of 0.26 or 1.20, respectively; substitution of Lys201 with Ala resulted in a relative rate of 0.12; and substitution of Met202 with Ala or norleucine resulted in a relative rate of 0.38 or 1.20, respectively. See Schmidt and Bostian, supra, 1997. These results indicate that a variety of residues can be substituted in a clostridial toxin substrate as compared to a naturally occurring toxin-sensitive sequence. In the case of BoNT/A, these results indicate that residues including but not limited to Glu194, Ala195, Gln197, Ala199, Thr200 and Met202, Leu203, Gly204, Ser205, and Gly206, as well as residues more distal from the Gln-Arg scissile bond, can be substituted or conjugated to a fluorophore, bulking group, donor fluorophore or acceptor in a BoNT/A substrate useful in the invention. Such a BoNT/A substrate is detectably proteolyzed at the scissile bond by BoNT/A under conditions suitable for clostridial toxin protease activity. Thus, a BoNT/A substrate can include, if desired, one or several amino acid substitutions, additions or deletions relative to a naturally occurring SNAP-25 sequence.

TABLE C

KINETIC PARAMETERS OF BONT/A SYNTHETIC
PEPTIDE SUBSTRATES

| | | | |
|---|---|---|---|
| | | SEQ ID | Relative |
| Peptide | Sequence^{a} | NO: | Rate^{b} |

| | | | |
|---|---|---|---|
| [1-15] | SNKTRIDEANQRATK | 31 | 0.03 |
| [1-16[ | SNIKTRIDEANQRATKM | 32 | 1.17 |
| [1-17] | SNKTRIDEANQRATKML | 33 | 1.00 |
| M16A | SNKTRIDEANQRATKAL | 50 | 0.38 |
| M16X | SNKTRTDEANQRATKXL | 51 | 1.20 |
| K15A | SNKTRIDEANQRATAML | 52 | 0.12 |
| T14S | SNKTRIDEANQRASKML | 53 | 0.26 |
| T14B | SNKTRIDEANQRAB KML | 54 | 1.20 |
| A13B | SNKTRIDEANQRBTKML | 55 | 0.79 |
| Q11A | SNKTRIDEANARATKML | 56 | 0.19 |
| Q11B | SNKTRIDEANBRATKML | 57 | 0.25 |
| Q11N | SNKTRIDEANNRATKML | 58 | 0.66 |
| N10A | SNKTRIDEAAQRATKML | 59 | 0.06 |
| A9B | SNKTRIDEBNQRATKML | 60 | 0.38 |
| E8Q | SNKTRIDQANQRATKML | 61 | 2.08 |
| D7N | SNKTRINEANQRATKML | 62 | 0.23 |

^{a}Nonstandard amino acid abbreviations are: B, 2-aminobutyric acid; X, 2-aminohexanoic acid (norleucine)
^{b}Initial hydrolysis rates relative to peptide [1-17]. Peptide concentrations were 1.0 mM.

### BoNT/B Recognition Sequences

As used herein, the term “botulinum toxin serotype B recognition sequence” is synonymous with “BoNT/B recognition sequence” and means a scissile bond together with adjacent or non-adjacent recognition elements, or both, sufficient for detectable proteolysis at the scissile bond by a BoNT/B under appropriate conditions. A scissile bond cleaved by BoNT/B can be, for example, Gln-Phe.

A variety of BoNT/B recognition sequences are well known in the art or can be defined by routine methods. Such BoNT/B recognition sequences can include, for example, a sequence corresponding to some or all of the hydrophilic core of a VAMP protein such as human VAMP-1 or human VAMP-2. A BoNT/B recognition sequence can include, without limitation, residues 33 to 94, residues 45 to 94, residues 55 to 94, residues 60 to 94, residues 65 to 94, residues 60 to 88 or residues 65 to 88 of human VAMP-2 (SEQ ID NO: 8), or residues 60 to 94 of human VAMP-1 (SEQ ID NO: 7). See, for example, Shone et al., Eur. J. Biochem. 217: 965-971(1993), and U.S. Pat. No. 5,962,637. If desired, a similar BoNT/B recognition sequence can be prepared from a corresponding (homologous) segment of another BoNT/B-sensitive VAMP isoform or homolog such as human VAMP-1 or rat or chicken VAMP-2.

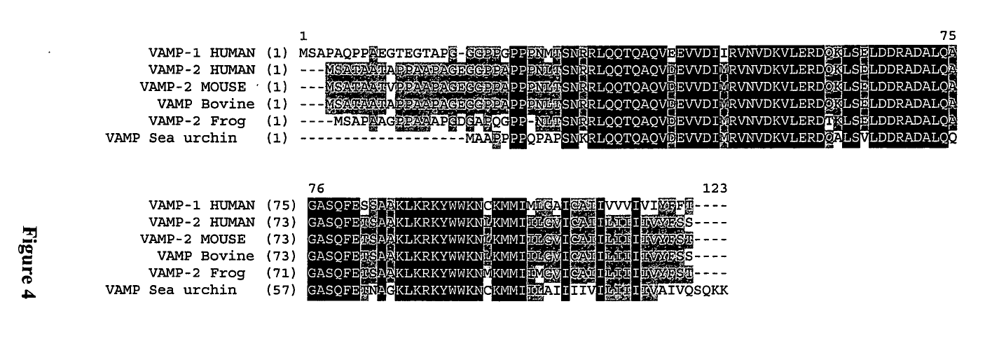

Thus, it is understood that a BoNT/B recognition sequence can correspond to a segment of a protein that is sensitive to cleavage by botulinum toxin serotype B, or can be substantially similar to such a segment of a BoNT/B-sensitive protein. As shown in Table D, a variety of naturally occurring proteins sensitive to cleavage by BoNT/B are known in the art and include, for example, human, mouse and bovine VAMP-1 and VAMP-2; rat VAMP-2; rat cellubrevin; chicken VAMP-2; Torpedo VAMP-1; sea urchin VAMP; Aplysia VAMP; squid VAMP; C. elegans VAMP; Drosophila n-syb; and leech VAMP. Thus, a BoNT/B recognition sequence included in a BoNT/B substrate can correspond, for example, to a segment of human VAMP-1 or VAMP-2, mouse VAMP-1 or VAMP-2, bovine VAMP-1 or VAMP-2, rat VAMP-2, rat cellubrevin, chicken VAMP-2, Torpedo VAMP-1, sea urchin VAMP, Aplysia VAMP, squid VAMP, C. elegans VAMP, Drosophila n-syb, leech VAMP, or another naturally occurring protein sensitive to cleavage by BoNT/B. Furthermore, as shown in Table D, comparison of native VAMP amino acid sequences cleaved by BoNT/B reveals that such sequences are not absolutely conserved (see, also, FIG. 4), indicating that a variety of amino acid substitutions and modifications relative to a naturally occurring VAMP sequence can be tolerated in a BoNT/B substrate of the invention.

TABLE D
Cleavage of VAMP^{a,b}

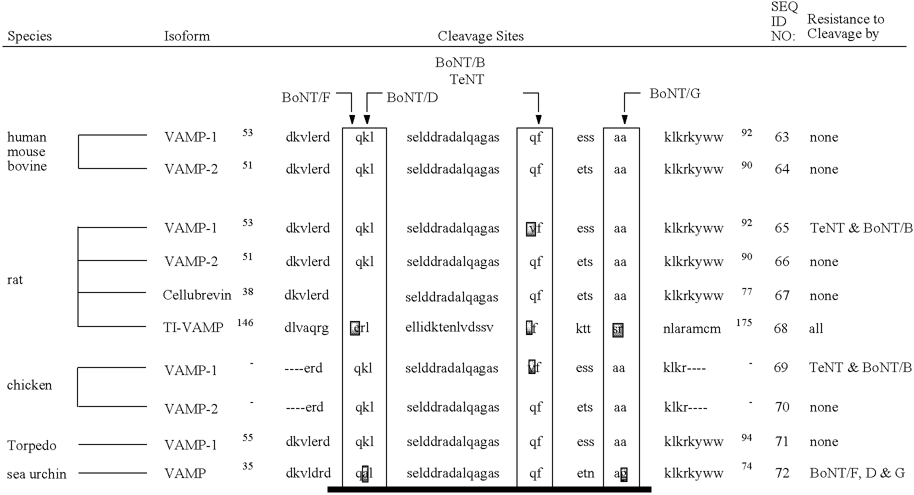

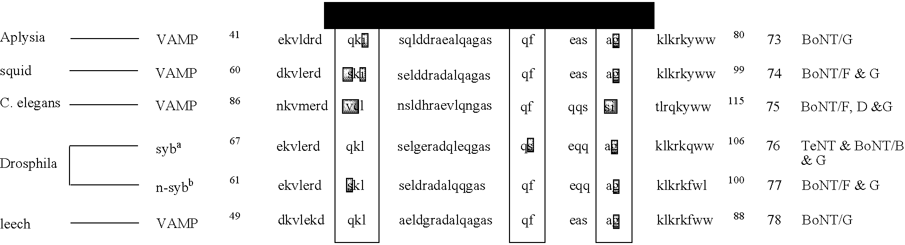

^{a}= Sequence corrected in position 93 (f > s).
^{b}= Sequence corrected in position 68 (t > s).

### BoNT/C1 Recognition Sequences

As used herein, the term “botulinum toxin serotype C1 recognition sequence” is synonymous with “BoNT/C1 recognition sequence” and means a scissile bond together with adjacent or non-adjacent recognition elements, or both, sufficient for detectable proteolysis at the scissile bond by a BoNT/C1 under appropriate conditions. A scissile bond cleaved by BoNT/C1 can be, for example, Lys-Ala or Arg-Ala.

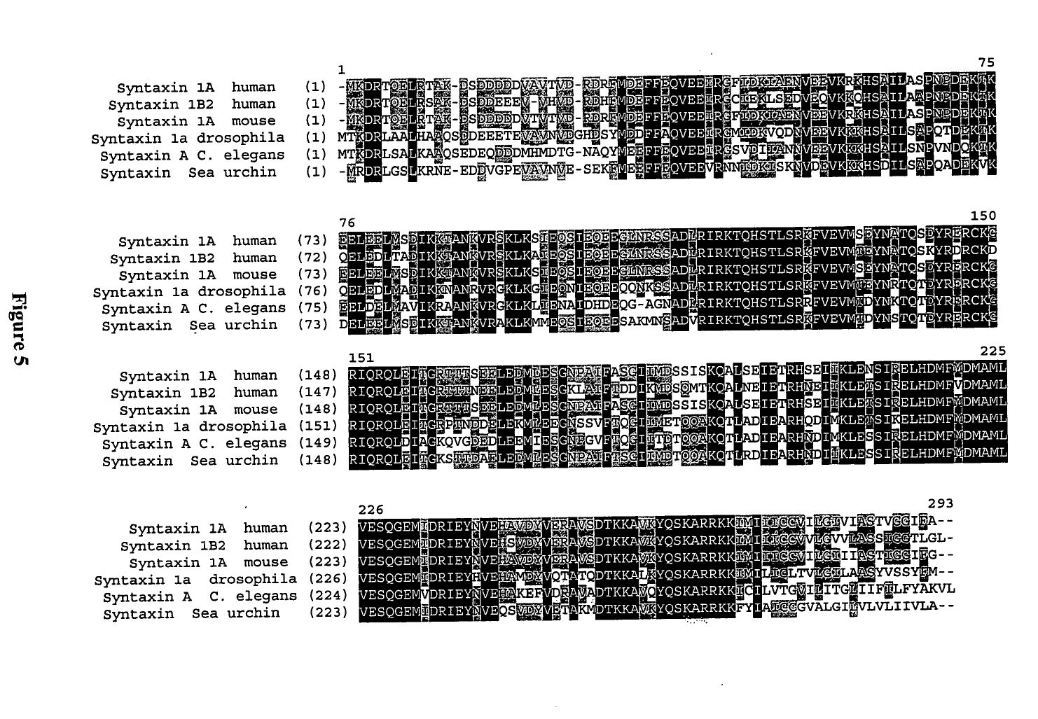

It is understood that a BoNT/C1 recognition sequence can correspond to a segment of a protein that is sensitive to cleavage by botulinum toxin serotype C1, or can be substantially similar to a segment of a BoNT/C1-sensitive protein. As shown in Table E, a variety of naturally occurring proteins sensitive to cleavage by BoNT/C1 are known in the art and include, for example, human, rat, mouse and bovine syntaxin 1A and 1B; rat syntaxins 2 and 3; sea urchin syntaxin; Aplysia syntaxin 1; squid syntaxin; Drosophila Dsynt1; and leech syntaxin 1. Thus, a BoNT/C1 recognition sequence useful in a BoNT/C1 substrate can correspond, for example, to a segment of human, rat, mouse or bovine syntaxin 1A or 1B, rat syntaxin 2, rat syntaxin 3, sea urchin syntaxin, Aplysia syntaxin 1, squid syntaxin, Drosophila Dsynt1, leech syntaxin 1, or another naturally occurring protein sensitive to cleavage by BoNT/C1. Furthermore, comparison of native syntaxin amino acid sequences cleaved by BoNT/C1 reveals that such sequences are not absolutely conserved (see Table E and FIG. 5), indicating that a variety of amino acid substitutions and modifications relative to a naturally occurring BoNT/C1-sensitive syntaxin sequence can be tolerated in a BoNT/C1 substrate useful in the invention.

TABLE E
Cleavage of syntaxin

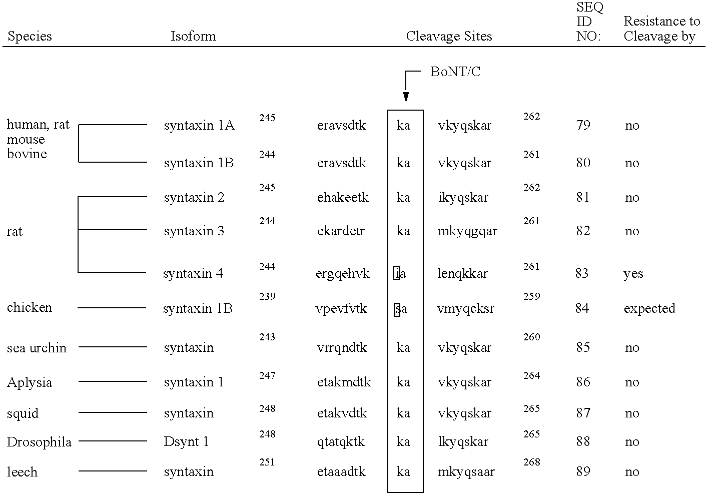

A variety of naturally occurring SNAP-25 proteins also are sensitive to cleavage by BoNT/C1, including human, mouse and rat SNAP-25; goldfish SNAP-25A and 25B; and Drosophila and leech SNAP-25. Thus, a BoNT/C1 recognition sequence useful in a BoNT/C1 substrate can correspond, for example, to a segment of human, mouse or rat SNAP-25, goldfish SNAP-25A or 25B, Torpedo SNAP-25, zebrafish SNAP-25, Drosophila SNAP-25, leech SNAP-25, or another naturally occurring protein sensitive to cleavage by BoNT/C1. As discussed above in regard to variants of naturally occurring syntaxin sequences, comparison of native SNAP-25 amino acid sequences cleaved by BoNT/C1 reveals significant sequence variability (see FIG. 3 and Table B above), indicating that a variety of amino acid substitutions and modifications relative to a naturally occurring BoNT/C1-sensitive SNAP-25 sequence can be tolerated in a BoNT/C1 substrate useful in the invention.

### BoNT/D Recognition Sequences

The term “botulinum toxin serotype D recognition sequence” is synonymous with “BoNT/D recognition sequence” and means a scissile bond together with adjacent or non-adjacent recognition elements, or both, sufficient for detectable proteolysis at the scissile bond by a BoNT/D under appropriate conditions. A scissile bond cleaved by BoNT/D can be, for example, Lys-Leu.

A variety of BoNT/D recognition sequences are well known in the art or can be defined by routine methods. A BoNT/D recognition sequence can include, for example, residues 27 to 116; residues 37 to 116; residues 1 to 86; residues 1 to 76; or residues 1 to 69 of rat VAMP-2 (SEQ ID NO: 90; Yamasaki et al., J. Biol. Chem. 269:12764-12772 (1994)). Thus, a BoNT/D recognition sequence can include, for example, residues 27 to 69 or residues 37 to 69 of rat VAMP-2 (SEQ ID NO: 90). If desired, a similar BoNT/D recognition sequence can be prepared from a corresponding (homologous) segment of another BoNT/D-sensitive VAMP isoform or homolog such as human VAMP-1 or human VAMP-2.

A BoNT/D recognition sequence can correspond to a segment of a protein that is sensitive to cleavage by botulinum toxin serotype D, or can be substantially similar to a segment of a BoNT/D-sensitive protein. As shown in Table D, a variety of naturally occurring proteins sensitive to cleavage by BoNT/D are known in the art and include, for example, human, mouse and bovine VAMP-1 and VAMP-2; rat VAMP-1 and VAMP-2; rat cellubrevin; chicken VAMP-1 and VAMP-2; Torpedo VAMP-1; Aplysia VAMP; squid VAMP; Drosophila syb and n-syb; and leech VAMP. Thus, a BoNT/D recognition sequence can correspond, for example, to a segment of human VAMP-1 or VAMP-2, mouse VAMP-1 or VAMP-2, bovine VAMP-1 or VAMP-2, rat VAMP-1 or VAMP-2, rat cellubrevin, chicken VAMP-1 or VAMP-2, Torpedo VAMP-1, Aplysia VAMP, squid VAMP, Drosophila syb or n-syb, leech VAMP, or another naturally occurring protein sensitive to cleavage by BoNT/D. Furthermore, as shown in Table D above, comparison of native VAMP amino acid sequences cleaved by BoNT/D reveals significant sequence variability (see, also, FIG. 4), indicating that a variety of amino acid substitutions and modifications relative to a naturally occurring BoNT/D-sensitive VAMP sequence can be tolerated in a BoNT/D substrate useful in the invention.

### BoNT/E Recognition Sequences

As used herein, the term “botulinum toxin serotype E recognition sequence” is synonymous with “BoNT/E recognition sequence” and means a scissile bond together with adjacent or non-adjacent recognition elements, or both, sufficient for detectable proteolysis at the scissile bond by a BoNT/E under appropriate conditions. A scissile bond cleaved by BoNT/E can be, for example, Arg-Ile.

One skilled in the art appreciates that a BoNT/E recognition sequence can correspond to a segment of a protein that is sensitive to cleavage by botulinum toxin serotype E, or can be substantially similar to a segment of a BoNT/E-sensitive protein. In one embodiment, a BoNT/E recognition sequence includes residues 134 to 206 of SEQ ID NO: 2. A variety of naturally occurring proteins sensitive to cleavage by BoNT/E are known in the art and include, for example, human, mouse and rat SNAP-25; mouse SNAP-23; chicken SNAP-25; goldfish SNAP-25A and SNAP-25B; zebrafish SNAP-25; C. elegans SNAP-25; and leech SNAP-25 (see Table B). Thus, a BoNT/E recognition sequence can correspond, for example, to a segment of human SNAP-25, mouse SNAP-25, rat SNAP-25, mouse SNAP-23, chicken SNAP-25, goldfish SNAP-25A or 25B, C. elegans SNAP-25, leech SNAP-25, or another naturally occurring protein sensitive to cleavage by BoNT/E. Furthermore, as shown in Table B and FIG. 3 above, comparison of native SNAP-23 and SNAP-25 amino acid sequences cleaved by BoNT/E reveals that such sequences are not absolutely conserved, indicating that a variety of amino acid substitutions and modifications relative to a naturally occurring BoNT/E-sensitive SNAP-23 or SNAP-25 sequence can be tolerated in a BoNT/E substrate useful in the invention.

### BoNT/F Recognition Sequences

The term “botulinum toxin serotype F recognition sequence,” as used herein, is synonymous with “BoNT/F recognition sequence” and means a scissile bond together with adjacent or non-adjacent recognition elements, or both, sufficient for detectable proteolysis at the scissile bond by a BoNT/F under appropriate conditions. A scissile bond cleaved by BoNT/F can be, for example, Gln-Lys.

A variety of BoNT/F recognition sequences are well known in the art or can be defined by routine methods. A BoNT/F recognition sequence can include, for example, residues 27 to 116; residues 37 to 116; residues 1 to 86; residues 1 to 76; or residues 1 to 69 of rat VAMP-2 (SEQ ID NO: 90; Yamasaki et al., supra, 1994). A BoNT/F recognition sequence also can include, for example, residues 27 to 69 or residues 37 to 69 of rat VAMP-2 (SEQ ID NO: 90). It is understood that a similar BoNT/F recognition sequence can be prepared, if desired, from a corresponding (homologous) segment of another BoNT/F-sensitive VAMP isoform or homolog such as human VAMP-1 or human VAMP-2.

A BoNT/F recognition sequence can correspond to a segment of a protein that is sensitive to cleavage by botulinum toxin serotype F, or can be substantially similar to a segment of a BoNT/F-sensitive protein. A variety of naturally occurring proteins sensitive to cleavage by BoNT/F are known in the art and include, for example, human, mouse and bovine VAMP-1 and VAMP-2; rat VAMP-1 and VAMP-2; rat cellubrevin; chicken VAMP-1 and VAMP-2; Torpedo VAMP-1; Aplysia VAMP; Drosophila syb; and leech VAMP (see Table D). Thus, a BoNT/F recognition sequence can correspond, for example, to a segment of human VAMP-1 or VAMP-2, mouse VAMP-1 or VAMP-2, bovine VAMP-1 or VAMP-2, rat VAMP-1 or VAMP-2, rat cellubrevin, chicken VAMP-1 or VAMP-2, Torpedo VAMP-1, Aplysia VAMP, Drosophila syb, leech VAMP, or another naturally occurring protein sensitive to cleavage by BoNT/F. Furthermore, as shown in Table D above, comparison of native VAMP amino acid sequences cleaved by BoNT/F reveals that such sequences are not absolutely conserved (see, also, FIG. 4), indicating that a variety of amino acid substitutions and modifications relative to a naturally occurring BoNT/F-sensitive VAMP sequence can be tolerated in a BoNT/F substrate useful in the invention.

### BoNT/G Recognition Sequences

As used herein, the term “botulinum toxin serotype G recognition sequence” is synonymous with “BoNT/G recognition sequence” and means a scissile bond together with adjacent or non-adjacent recognition elements, or both, sufficient for detectable proteolysis at the scissile bond by a BoNT/G under appropriate conditions. A scissile bond cleaved by BoNT/G can be, for example, Ala-Ala.

A BoNT/G recognition sequence can correspond to a segment of a protein that is sensitive to cleavage by botulinum toxin serotype G, or can be substantially similar to such a BoNT/G-sensitive segment. As illustrated in Table D above, a variety of naturally occurring proteins sensitive to cleavage by BoNT/G are known in the art and include, for example, human, mouse and bovine VAMP-1 and VAMP-2; rat VAMP-1 and VAMP-2; rat cellubrevin; chicken VAMP-1 and VAMP-2; and Torpedo VAMP-1. Thus, a BoNT/G recognition sequence can correspond, for example, to a segment of human VAMP-1 or VAMP-2, mouse VAMP-1 or VAMP-2, bovine VAMP-1 or VAMP-2, rat VAMP-1 or VAMP-2, rat cellubrevin, chicken VAMP-1 or VAMP-2, Torpedo VAMP-1, or another naturally occurring protein sensitive to cleavage by BoNT/G. Furthermore, as shown in Table D above, comparison of native VAMP amino acid sequences cleaved by BoNT/G reveals that such sequences are not absolutely conserved (see, also, FIG. 4), indicating that a variety of amino acid substitutions and modifications relative to a naturally occurring BoNT/G-sensitive VAMP sequence can be tolerated in a BoNT/G substrate useful in the invention.

### TeNT Recognition Sequences

As used herein, the term “tetanus toxin recognition sequence” means a scissile bond together with adjacent or non-adjacent recognition elements, or both, sufficient for detectable proteolysis at the scissile bond by a tetanus toxin under appropriate conditions. A scissile bond cleaved by TeNT can be, for example, Gln-Phe.

A variety of TeNT recognition sequences are well known in the art or can be defined by routine methods and include sequences corresponding to some or all of the hydrophilic core of a VAMP protein such as human VAMP-1 or human VAMP-2. A TeNT recognition sequence can include, for example, residues 25 to 93 or residues 33 to 94 of human VAMP-2 (SEQ ID NO: 8; Comille et al., Eur. J. Biochem. 222:173-181(1994); Foran et al., Biochem. 33: 15365-15374 (1994)); residues 51 to 93 or residues 1 to 86 of rat VAMP-2 (SEQ ID NO: 90; Yamasaki et al., supra, 1994); or residues 33 to 94 of human VAMP-1 (SEQ ID NO: 7). A TeNT recognition sequence also can include, for example, residues 25 to 86, residues 33 to 86 or residues 51 to 86 of human VAMP-2 (SEQ ID NO: 8) or rat VAMP-2 (SEQ ID NO: 90). It is understood that a similar TeNT recognition sequence can be prepared, if desired, from a corresponding (homologous) segment of another TeNT-sensitive VAMP isoform or species homolog such as human VAMP-1 or sea urchin or Aplysia VAMP.

Thus, a TeNT recognition sequence can correspond to a segment of a protein that is sensitive to cleavage by tetanus toxin, or can be substantially similar to a segment of a TeNT-sensitive protein. As shown in Table D above, a variety of naturally occurring proteins sensitive to cleavage by TeNT are known in the art and include, for example, human, mouse and bovine VAMP-1 and VAMP-2; rat VAMP-2; rat cellubrevin; chicken VAMP-2; Torpedo VAMP-1; sea urchin VAMP; Aplysia VAMP; squid VAMP; C. elegans VAMP; Drosophila n-syb; and leech VAMP. Thus, a TeNT recognition sequence can correspond, for example, to a segment of human VAMP-1 or VAMP-2, mouse VAMP-1 or VAMP-2, bovine VAMP-1 or VAMP-2, rat VAMP-2, rat cellubrevin, chicken VAMP-2, Torpedo VAMP-1, sea urchin VAMP, Aplysia VAMP, squid VAMP, C. elegans VAMP, Drosophila n-syb, leech VAMP, or another naturally occurring protein sensitive to cleavage by TeNT. Furthermore, comparison of native VAMP amino acid sequences cleaved by TeNT reveals that such sequences are not absolutely conserved (Table D and FIG. 4). This finding indicates that a variety of amino acid substitutions and modifications relative to a naturally occurring TeNT-sensitive VAMP sequence can be tolerated in a TeNT substrate useful in the invention.

The clostridial toxin substrates of the invention include peptides and peptidomimetics as well as derivatized forms thereof. As used herein, the term “peptidomimetic” is used broadly to mean a peptide-like molecule that is cleaved by the same clostridial toxin as the peptide substrate upon which it is structurally based. Such peptidomimetics include chemically modified peptides, peptide-like molecules containing non-naturally occurring amino acids, and peptoids, which are peptide-like molecules resulting from oligomeric assembly of N-substituted glycines, and are cleaved by the same clostridial toxin as the peptide substrate upon which the peptidomimetic is derived (see, for example, Goodman and Ro, Peptidomimetics for Drug Design, in “Burger's Medicinal Chemistry and Drug Discovery” Vol. 1 (ed. M. E. Wolff; John Wiley & Sons 1995), pages 803-861).

A variety of peptidomimetics are known in the art including, for example, peptide-like molecules which contain a constrained amino acid, a non-peptide component that mimics peptide secondary structure, or an amide bond isostere. A peptidomimetic that contains a constrained, non-naturally occurring amino acid can include, for example, an α-methylated amino acid; an α,α-dialkyl-glycine or α-aminocycloalkane carboxylic acid; an N^{α}—C^{α} cylized amino acid; an N^{α}-methylated amino acid; a β- or γ-amino cycloalkane carboxylic acid; an α,β-unsaturated amino acid; a β,β-dimethyl or β-methyl amino acid; a β-substituted -2,3-methano amino acid; an NC⁶⁷ or C^{α—C}⁶⁷ cyclized amino acid; or a substituted proline or another amino acid mimetic. In addition, a peptidomimetic which mimics peptide secondary structure can contain, for example, a nonpeptidic β-turn mimic; γ-turn mimic; mimic of β-sheet structure; or mimic of helical structure, each of which is well known in the art. A peptidomimetic also can be a peptide-like molecule which contains, for example, an amide bond isostere such as a retro-inverso modification; reduced amide bond; methylenethioether or methylenesulfoxide bond; methylene ether bond; ethylene bond; thioamide bond; trans-olefin or fluoroolefin bond; 1,5-disubstituted tetrazole ring; ketomethylene or fluoroketomethylene bond or another amide isostere. One skilled in the art understands that these and other peptidomimetics are encompassed within the meaning of the term “peptidomimetic” as used herein.

In any of the methods of the invention, a clostridial toxin substrate can include one or multiple clostridial toxin cleavage sites for the same or different clostridial toxins. In particular embodiments, the invention provides methods that rely on a clostridial toxin substrate which contains a single clostridial toxin cleavage site. In other embodiments, the invention provides methods which rely on a clostridial toxin substrate which contains multiple cleavage sites for the same clostridial toxin. These cleavage sites can be incorporated within identical or different clostridial toxin recognition sequences. As non-limiting examples, a clostridial toxin substrate can have multiple cleavage sites for the same clostridial toxin intervening between the same lanthanide donor complex and acceptor. A clostridial toxin substrate useful in the invention can contain, for example, two or more, three or more, five or more, or ten or more identical or non-identical recognition sequences for the same clostridial toxin. A clostridial toxin substrate useful in the invention also can have, for example, two, three, four, five, six, seven, eight, nine or ten recognition sequences for the same clostridial toxin; the multiple recognition sequences can intervene between the same or different lanthanide donor complex-acceptor pairs.

A clostridial toxin substrate useful in the invention also can include cleavage sites for different clostridial toxins. In particular embodiments, the invention provides a method that relies on a clostridial toxin substrate which includes multiple cleavage sites for different clostridial toxins all intervening between the same lanthanide donor complex and acceptor. A clostridial toxin substrate can include, for example, cleavage sites for two or more, three or more, or five or more different clostridial toxins all intervening between the same lanthanide donor complex and acceptor. A clostridial toxin substrate also can incorporate, for example, cleavage sites for two or more, three or more, or five or more different clostridial toxins intervening between at least two lanthanide donor complex-acceptor pairs. In particular embodiments, the invention provides a clostridial toxin substrate having cleavage sites for two, three, four, five, six, seven or eight different clostridial toxins, where the cleavage sites intervene between the same or different lanthanide donor complex-acceptor pairs. In further embodiments, the invention provides a clostridial toxin substrate which has any combination of two, three, four, five, six, seven or eight cleavage sites for any combination of the following clostridial toxins: BoNT/A, BoNT/B, BoNT/C1, BoNT/D, BoNT/E, BoNT/F, BoNT/G and TeNT.

A method of the invention optionally can be performed with multiple substrates. In such a method, a first clostridial toxin substrate is treated with a sample, the first substrate including a first lanthanide donor complex, a first acceptor having an absorbance spectrum which overlaps the emission spectrum of the first lanthanide donor complex, and a first clostridial toxin recognition sequence containing a cleavage site, where the cleavage site intervenes between the first lanthanide donor complex and the first acceptor and where, under the appropriate conditions, resonance energy transfer is exhibited between the first lanthanide donor complex and the first acceptor. If desired, a second clostridial toxin substrate can be included in the same assay; this second substrate contains a second lanthanide donor complex and second acceptor having an absorbance spectrum which overlaps the emission spectrum of the second lanthanide donor complex, and a second clostridial toxin recognition sequence that is cleaved by a different clostridial toxin than the toxin that cleaves the first clostridial toxin recognition sequence. The lanthanide donor complex-acceptor pair in the second substrate can be the same or different from the lanthanide donor complex-acceptor pair in the first substrate. In this way, a single sample can be simultaneously assayed for the presence of more than one clostridial toxin.

It is understood that one can use a method of the invention to assay for any combination of clostridial toxins, for example, two, three, four, five, six, seven, eight, or more clostridial toxins. One can assay, for example, any combination of two, three, four, five, six, seven or eight of BoNT/A, BoNT/B, BoNT/C1, BoNT/D, BoNT/E, BoNT/F, BoNT/G and TeNT. As an example, an assay can be performed with seven substrates, each of which includes GFP and CS124-DTPA-EMCH—Tb flanking, respectively, a BoNT/A, BoNT/B, BoNT/C1, BoNT/D, BoNT/E, BoNT/F or BoNT/G recognition sequence and cleavage site. These substrates can be treated with a sample under conditions suitable for botulinum toxin activity before exciting carbostyryl 124 at 330 nm and monitoring terbium emission at 586 nm. An increase in luminescence intensity at 586 nm (relief of quenching) is indicative of the presence or activity of at least one clostridial toxin. Such an assay can be useful, for example, for assaying food samples or tissue samples for the presence of any botulinum or other clostridial toxin and can be combined, if desired, with one or more subsequent assays for individual clostridial toxins or specific combinations of clostridial toxins.

It further is understood that a single sample can be assayed for two or more different clostridial toxins using two or more different clostridial toxin substrates, with each substrate containing a different lanthanide donor complex-acceptor pair. The use of multiple substrates can be useful for extending the dynamic range of an assay, as described, for example, in U.S. Pat. No. 6,180,340. Those skilled in the art understand that the first antenna in the first lanthanide donor complex can be excited before or after excitation of the second antenna in the second lanthanide donor complex, and that the change in resonance energy transfer of the first substrate can be determined before, at the same time, or after determining resonance energy transfer of the second substrate.

The following examples are intended to illustrate but not limit the present invention.

### EXAMPLE I

### Preparation of Lanthanide-Based Substrates

This example describes construction of substrates containing a terbium or other lanthanide ion suitable for assaying for the presence or activity of a clostridial toxin.

### A. Construction of GFP-SNAP25₍₁₃₄₋₂₀₆₎₋His6-C

A substrate was prepared as a fusion protein containing green fluorescent protein (GFP), murine SNAP-25 residues 134-206, a polyhistidine affinity tag (6xHis), and a carboxy-terminal cysteine, with several components separated by peptide linkers. As described further below, the substrate was designed such that the GFP and terminal cysteine were present at opposite ends of SNAP-25₍₁₃₄₋₂₀₆₎.

The SNAP-25 sequence was obtained from pT25FL, a plasmid which contains the full-length mouse SNAP-25 gene inserted in frame with the 3′ terminus of the glutathione-S-transferase (GST) gene (GST-SNAP25₍₁₋₂₀₆₎), provided by Professor Dolly (O'Sullivan et al., J. Biol. Chem. 274:36897-36904 (1999)). The SNAP-25 sequence from pT25FL was incorporated into a second expression vector, which was designed to have a BirAsp signal sequence for biotinylation and a polyhistidine affinity tag fused to the amino-terminus of residues 134 to 206 of SNAP-25 (BirAsp-polyHis-SNAP25₍₁₃₄₋₂₀₆₎, denoted “BA-SNAP”). The DNA sequence encoding SNAP25₍₁₃₄₋₂₀₆₎ was generated by PCR amplification of the appropriate region of the pT25FL plasmid with PCR primers 5′-GCT AGA TCT CGA GTT AAC CAC TTC CCA GCA TCT TTG-3′(SEQ ID NO: 91; antisense) and 5′-ATC CGG AGG GTA ACA AAC GAT GCC-3′(SEQ ID NO: 92; sense) to produce a SNAP25₍₁₃₄₋₂₀₆₎ PCR product containing a Bgl II restriction site (PCR product A).

The BirAsp sequence, a natural substrate for biotinylation, as well as a polyhistidine affinity tag, were engineered for fusion upstream and in frame with the SNAP25₍₁₃₄₋₂₀₆₎ sequence using synthetic oligonucleotides SEQ ID NOS: 93 and 94, which contained a 20 bp complementary region. These oligonucleotides, 5′-CGA ATT CCG CGG GCC ACC ATG GGA GGA GGA CTG AAC GAC ATC TTC GAG GCT CAA AAG ATC-3′(SEQ ID NO: 93; sense; Sac II site underlined) and 5′-TCG TTT GTTACC CTC CGG ATA TGA TGA TGA TGA TGA TGA TGA TGG GAT CCA TGC CAC TCG ATC TTT TGA GCC TCG AAG A-3′(SEQ ID NO: 94; antisense), were annealed, and the single strand overhangs filled by PCR amplification to yield PCR product B.

The two double stranded PCR products containing the coding sequences for SNAP25₍₁₃₄₋₂₀₆₎, denoted PCR product A, and BirAsp and polyhistidine, denoted PCR product B, were denatured and annealed. The 20 bp complementary sequence in the two gene fragments is shown in italics in PCR primers SEQ ID NO: 92 and SEQ ID NO: 94. After filling in the overhangs by PCR, the product was amplified with primers SEQ ID NO: 93 and SEQ ID NO: 91. The resulting PCR product, which encoded BirAsp-polyHis-SNAP25₍₁₃₄₋₂₀₆₎ (designated “BA-SNAP”), was digested with SacII and BglII, and the isolated gene insert ligated into pQBI25fA2 vector digested with SacII and BamHI, to yield plasmid pNTP12 (pQBI25fA2 containing BA-SNAP).

For expression and purification from E. coli, the BA-SNAP gene was transferred into a pTrc99A plasmid (Amersham Pharmacia Biotech). The BA-SNAP gene was isolated from pNTP12 by digestion with NcoI and XhoI followed by gel purification. Separately, the pTrc99A plasmid was digested with NcoI and SaII, and the isolated vector ligated to the BA-SNAP gene to yield plasmid pNTP14 (pTrc99A containing BA-SNAP).

For cloning of the BA-SNAP gene into plasmid pQE-50, the BA-SNAP fragment was PCR amplified from pNTP14 with primer SEQ ID NO: 91 and primer SEQ ID NO: 95 (5′-CGA AGA TCT GGA GGA CTG AAC GAC ATC TTC-3′(sense; Bgl II site underlined)). After digestion with BglII and XhoI, the amplified PCR product was ligated into vector pQE-50, which had been digested with BamH I and Sal I. The resulting plasmid, which represents pQE50 containing BA-SNAP, was designated pNTP26.

A plasmid encoding the green fluorescent protein (GFP) fusion protein substrate was prepared by modifying vector pQBI T7-GFP (Quantum Biotechnologies; Carlsbad, Calif.) in three phases as described below. First, vector PQBI T7-GFP was PCR-modified to remove the stop codon at the 3′ terminus of the GFP-coding sequence and to insert the coding sequence for a portion of the peptide linker separating GFP from the SNAP-25 fragment. Second, a DNA fragment coding for SNAP-25₍₁₃₄₋₂₀₆₎ was PCR amplified from pNTP26 using PCR primers designed to incorporate the coding sequence for the remainder of the peptide linker fused 5′ to the SNAP-25₍₁₃₄₋₂₀₆₎ gene and a 6xHis affinity tag fused 3′ of the gene. The resultant PCR product was cloned into the modified pQBI vector described above to yield pQBI GFP-SNAP25₍₁₃₄₋₂₀₆₎.

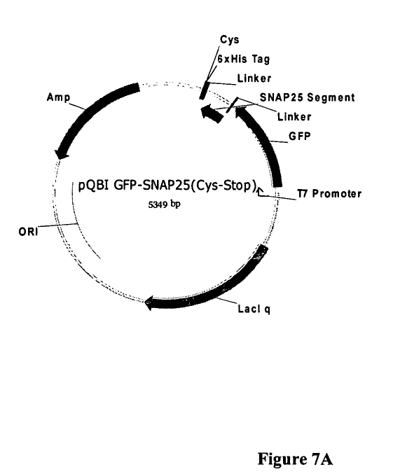

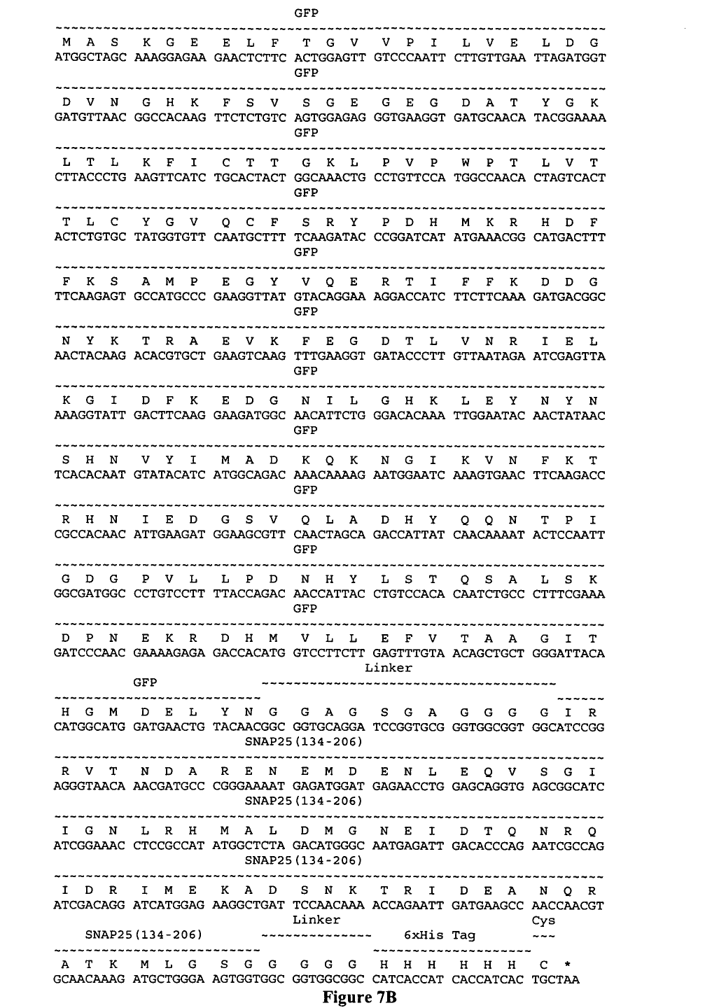

Plasmid pQBI GFP-SNAP25₍₁₃₄₋₂₀₆₎ was then modified by site-directed mutagenesis to add a cysteine codon at the carboxy-terminus using primer SEQ ID NO: 96 (5′-GATGGTGATGGTGATGACAGCCGCCACC GCCACC-3′ (antisense primer, with the added nucleotides underlined) and its reverse complement (sense primer). The resulting plasmid, designated pQBI GFP-SNAP25 (Cys-Stop), is shown in FIG. 7A and was used for expression of GFP-SNAP25₍₁₃₄₋₂₀₆₎-6xHis-Cys. The nucleic acid and predicted amino acid sequence for the GFP-SNAP25₍₁₃₄₋₂₀₆₎-6XHis-cysteine construct is shown herein in FIG. 7B.

### B. Expression and Characterization of GFP-SNAP25₍₁₃₄₋₂₀₆₎-His6-C

The pQBI GFP-SNAP25 (Cys-Stop) expression vector was transformed into E. coli BL21(DE3) cells (Novagen; Madison, Wis.; or Invitrogen; Carlsbad, Calif.) or into E. coli BL21-CodonPlus® (DE3)-RIL cells (Stratagene) containing the T7 RNA polymerase gene. Transformed cells were selected on LB-ampicillin plates overnight at 37° C. Single colonies were used to inoculate 1-3 mL starter cultures, which were in turn used to inoculate 0.5 to 1.0 L cultures. The large cultures were grown at 37° C. with shaking until A₅₉₅ reached 0.5-0.6, at which time they were removed from the incubator and were allowed to cool briefly. After induction of protein expression with 1 mM EPTG, GFP-SNAP25₍₁₃₄₋₂₀₆₎-His6-C substrate was expressed from the pQBI GFP-SNAP25 (Cys-Stop) plasmid overnight with shaking at 16° C. in order to facilitate formation of the GFP fluorophore. Cells from 250 mL aliquots of the expression cultures were collected by centrifugation (30 minutes, 6,000×g, 4° C.) and stored at −80° C. until needed.

Substrates were purified at 4° C. by a two-step procedure involving IMAC purification, followed by a de-salting step to remove NaCl and imidazole, typically yielding greater than 150 mg/L of purified substrate as follows. Cell pellets from 250 mL cultures were each resuspended in 7-12 mL Column Binding Buffer (25 mM HEPES, pH 8.0; 500 mM NaCl; 1 mM β-mercaptoethanol; 10 mM imidazole), lysed by sonication (1 minute 40 seconds in 10-second pulses at 38% amplitude), and clarified by centrifugation (16000 rpm, 4° C., 1 hour). Affinity resin (3-5 mL Talon SuperFlow Co²⁺ per cell pellet) was equilibrated in a glass or disposable column support (Bio-Rad) by rinsing with 4 column volumes of sterile ddH₂O and 4 column volumes of Column Binding Buffer. Clarified lysate was applied to the column in one of two ways: (1) Lysate was added to the resin and batch bound by horizontal incubation for 1 hour with gentle rocking or (2) Lysate was applied to the vertical column and allowed to enter the column slowly by gravity flow. Following batch binding only, the column was righted and the solution drained, collected, and passed over the resin again. In both cases, after the lysate had been applied, the column was washed with 4-5 column volumes of Column Binding Buffer. In some cases, the column was further washed with 1-2 column volumes of Column Wash Buffer (25 mM HEPES, pH 8.0; 500 mM NaCl; 1 mM β-mercaptoethanol; 20 mM imidazole). Protein was eluted with 1.5 to 2.0 column volumes of Column Elution Buffer (25 mM HEPES, pH 8.0; 500 mM NaCl; 1 mM β-mercaptoethanol; 250 mM imidazole), which was collected in fractions of 1.4 mL. The green fractions were combined, concentrated with a centrifugal filter (10,000 or 30,000 molecular weight cut-off) and desalted by FPLC (BioRad Biologic DuoLogic, QuadTec UV-Vis detector) with a HiPrep 26/10 size exclusion column (Pharmacia) and an isocratic mobile phase of chilled Fusion Protein Desalting Buffer (50 mM HEPES, pH 7.4, 4° C.) at a flow rate of 10 mL/minute. Desalted protein was collected as a single fraction, and the concentration determined using a BioRad Protein Assay. The GFP-SNAP25₍₁₃₄₋₂₀₆₎-His6-C substrate was analyzed by reducing SDS-PAGE. The protein solution was subsequently divided into 500 μL aliquots, flash-frozen with liquid nitrogen and stored at −80° C. Once defrosted, a working aliquot was stored at 4° C., protected from light.

### C. Labeling with Lumiphore CS124-DTPA-EMCH—Tb

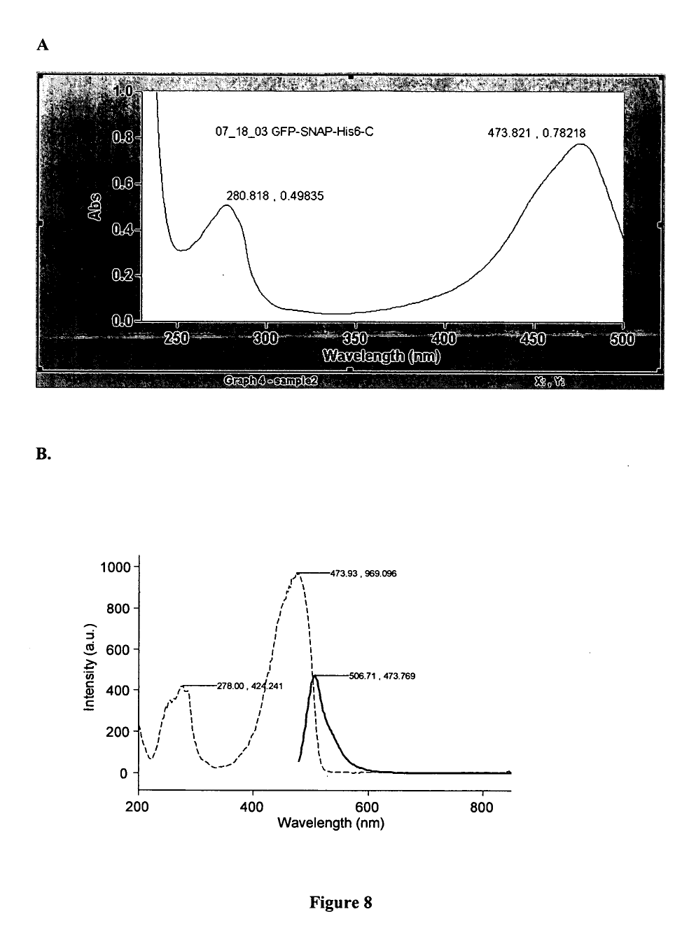

The GFP-SNAP25₍₁₃₄₋₂₀₆₎-His6-C construct contains a single cysteine residue which is solvent exposed, although there are three buried cysteine residues within GFP which are not available for chemical modification (Selvin, supra, 2000; Heyduk, Curr. Opin. Biotech. 13:292-296 (2002)). The carboxy-terminal cysteine residue can therefore be selectively labeled using a fluorophore-maleimide at neutral pH. Shown in FIGS. 8A and 8B, respectively, are the absorption and emission/excitation spectra of purified GFP-SNAP25₍₁₃₄₋₂₀₆₎-His6-C protein. The concentration of the protein solution was determined to be 2.74 mg/ml based on the theoretical molar extinction coefficient of 20250 M⁻¹ cm⁻¹ as calculated from the primary sequence of the construct. The molecular weight of the purified GFP-SNAP25₍₁₃₄₋₂₀₆₎-His6-C protein was confirmed to be about 37,000 using Matrix Assisted Laser Desorption Time of Flight mass spectrometry (MALDI-TOF).

The lumiphore CS124-DTPA-EMCH—Tb was obtained from Invitrogen Lifetechnologies (Carlsbad, Calif.), and GFP-SNAP25₍₁₃₄₋₂₀₆₎-His6-C-CS124-DTPA-EMCH—Tb was produced by derivatizing the carboxy-terminal cysteine of GFP-SNAP25₍₁₃₄₋₂₀₆₎-His6-C using maleimide chemistry at pH 6.9 in HEPES buffer. Unreacted probe was removed by extensive dialysis in 20 mM HEPES buffer pH 6.9 using a 25 kDa membrane. The absorption and emission spectra of the resulting CS124-DTPA-EMCH—Tb labeled GFP-SNAP25₍₁₃₄₋₂₀₆₎-His6-C are shown in FIGS. 9A and 9B, respectively.

### EXAMPLE II

### Assays for Clostridial Toxin Activity using Lanthanice-Based Substrates

This example describes the use of a lanthanide-based substrate for assaying BoNT/A activity.

Upon excitation of the sensitizing group carbostyryl 124 (CS124) at 330 nm, terbium produces a long lifetime emission in a series of four prominent sharp bands at 490 nm, 546 nm, 586 mn and 622 nm (see FIG. 9B). GFP absorbs maximally at 474 nm, with an emission maximum at 507 nm. Energy transfer was observed by monitoring Tb emission at 586 nm. As shown in FIG. 10A, there was a notable increase in luminescence intensity during the addition of reduced bulk BoNT-A toxin, indicative of the relief of quenching between the lanthanide donor complex and GFP. Furthermore, the signal to noise ratio for the emission process was enhanced by utilizing a gated process to monitor emission. By opening the emission gate for the emitted light after 200 μs, all the emission due to spurious fluorescent contaminants with lifetimes much shorter than the 10¹-10² μs lifetimes of the lanthanide probe was avoided. As shown in FIG. 10B, in which the dotted trace represents gated terbium emission before turnover of substrate and the solid trace represents gated terbium emission after turnover, the resulting gated signal was very clean and contributed to good levels of sensitivity.

These results indicate that GFP-SNAP25₍₁₃₄₋₂₀₆₎-His6-C can be derivatized with a commercially available lanthanide donor complex such as CS124-DTPA-EMCH—Tb to produce a clostridial toxin substrate which exhibits luminescence resonance energy transfer between the lanthanide donor complex and an acceptor such as GFP. The relief of quenching upon addition of BoNT/A reduced toxin was indicative of the activity of BoNT/A.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the following claims.

1. A clostridial toxin substrate, comprising:
(a) a lanthanide donor complex;
(b) an acceptor having an absorbance spectrum overlapping the emission spectrum of said lanthanide donor complex; and
(c) a clostridial toxin recognition sequence comprising a cleavage site,
wherein said cleavage site intervenes between said lanthanide donor complex and said acceptor and wherein, under the appropriate conditions, resonance energy transfer is exhibited between said lanthanide donor complex and said acceptor.

2. The substrate of claim 1, wherein said lanthanide donor complex has a fluorescence lifetime of at least 500 μs.

3. The substrate of claim 1, wherein said lanthanide donor complex has a fluorescence quantum yield of at least 0.05.

4. The substrate of claim 1, wherein said lanthanide donor complex has a fluorescence quantum yield of at least 0.5.

5. The substrate of claim 1, wherein said lanthanide donor complex comprises a lanthanide ion selected from the group of a terbium ion, europium ion, samarium ion and dysprosium ion.

6. The substrate of claim 5, wherein said lanthanide ion is a terbium ion.

7. The substrate of claim 1, wherein said lanthanide donor complex comprises a lanthanide-binding site which is a peptide or peptidomimetic.

8. The substrate of claim 7, wherein said lanthanide-binding site comprises the coordination site of an EF hand motif.

9. The substrate of claim 8, wherein said lanthanide-binding site comprises an EF hand motif.

10. The substrate of claim 7, wherein said lanthanide-binding site comprises a thiol-reactive chelator.

11. The substrate of claim 1, wherein said lanthanide donor complex comprises a lanthanide-binding site which comprises diethylenetriaminepentacetic acid (DTPA).

12. The substrate of claim 1, wherein said lanthanide donor complex comprises a lanthanide-binding site which is selected from the group of a β-diketone chelate, polyaminopolycarboxylic acid chelate, calixarene chelate, polyphenol, DOTA, pyridine and polypyridine.

13. The substrate of claim 1, wherein said lanthanide donor complex comprises a lanthanide-binding site which is selected from the group trisbipyridine (TBP) cryptate; trisbipyridine tetracarboxylate (TBP4COOH) cryptate; trisbipyridine pentacarboxylate (TBP5COOH) cryptate; and pyridine bipyridine tetracarboxylate (PBP4COOH).

14. The substrate of claim 1, wherein said lanthanide donor complex comprises a lanthanide-binding site which has an affinity for a lanthanide ion of at least 5 μM.

15. The substrate of claim 1, 6 or 8, wherein said lanthanide donor complex comprises an antenna which is a tryptophan residue.

16. The substrate of claim 1, wherein said lanthanide donor complex comprises an antenna which is selected from the group carbostyryl124 (CS124), tryptophan and 2-hydroxyisophthalamide.

17. The substrate of claim 16, wherein said antenna is carbostyryl124 (CS124).

18. The substrate of claim 11, wherein said lanthanide donor complex is CS124-DTPA-EMCH—Tb.

19. The substrate of claim 1, wherein said acceptor is an acceptor fluorophore.

20. The substrate of claim 1 or claim 18, wherein said acceptor is selected from the group green fluorescent protein (GFP), blue fluorescent protein (BFP), yellow fluorescent protein (YFP), cyan fluorescent protein (CFP) and red fluorescent protein (RFP).

21. The substrate of claim 20, wherein said acceptor is GFP.

22. The substrate of claim 1, wherein said acceptor is a non-fluorescent acceptor.

23. The substrate of claim 22, wherein said non-fluorescent acceptor is a heme protein.

24. The substrate of claim 1, comprising a botulinum toxin recognition sequence.

25. The substrate of claim 24, wherein said recognition sequence is a BoNT/A recognition sequence.

26. The substrate of claim 25, wherein said BoNT/A recognition sequences comprises at least six consecutive residues of SNAP-25, said six consecutive residues comprising Gln-Arg, or a peptidomimetic thereof.

27-41. (canceled)

42. The substrate of claim 1, 6 or 7, which is a peptide or peptidomimetic having at most 300 residues.

43. The substrate of claim 1, 6 or 7, which is a peptide or peptidomimetic having at most 150 residues.

44. The substrate of claim 1, wherein said substrate can be cleaved with an activity of at least 1 nanomole/minute/milligram toxin.

45. The substrate of claim 1, wherein said substrate can be cleaved with an activity of at least 20 nanomoles/minute/milligram toxin.

46. The substrate of claim 1, wherein said substrate can be cleaved with an activity of at least 100 nanomoles/minute/milligram toxin.

47-130. (canceled)


SEQUENCE LISTING (101 sequences)

SEQ ID NO: 1 - protein, 206 aa, Homo sapiens
MAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 2 - protein, 206 aa, Mus musculus; identical to SEQ ID NO: 1

SEQ ID NO: 3 - protein, 212 aa, Drosophila sp.
MPADPSEEVAPQVPKTELEELQINAQGVADESLESTRRMLALCEESKEAGIRTLVALDDQGEQLDRIEEGMDQINADMREAEKNLSGMEKCCGICVLPCNKSQSFKEDDGTWKGNDDGKVVNNQPQRVMDDRNGMMAQAGYIGRITNDAREDEMEENMGQVNTMIGNLRNMALDMGSELENQNRQIDRINRKGESNEARIAVANQRAHQLLK

SEQ ID NO: 4 - protein, 203 aa, Carassius auratus
MADEADMRNELTDMQARADQLGDESLESTRRMLQLVEESKDAGIRTLVMLDEQGEQLERIEEGMDQINKDMKEAEKNLTDLGNLCGLCPCPCNKLKGGGQSWGNNQDGVVSSQPARVVDEREQMAISGGFIRRVTNDARENEMDENLEQVGSIIGNLRHMALDMGNEIDTQNRQIDRIMDMADSNKTRIDEANQRATKMLGSG

SEQ ID NO: 5 - protein, 212 aa, Strongylcentrotas purpuratus
MEDQNDMNMRSELEEIQMQSNMQTDESLESTRRMLQMAEESQDMGIKTLVMLDEQGEQLDRIEEGMDQINTDMREAEKNLTGLEKCCGICVCPWKKLGNFEKGDDYKKTWKGNDDGKVNSHQPMRMEDDRDGCGGNASMITRITNDAREDEMDENLTQVSSIVGNLRHMAIDMQSEIGAQNSQVGRITSKAESNEGRINSADKRAKNILRNK

SEQ ID NO: 6 - protein, 249 aa, Gallus gallus
MAEDADMRNELEEMQRRADQLADESLESTRRMLQLVEESKDAGIRTLVMLDEQGEQLDRVEEGMNHINQDMKEAEKNLKDLGKCCGLFICPCNKLKSSDAYKKAWGNNQDGVVASQPARVVDEREQMAISGGFIRRVTNDARENEMDENLEQVSGIIGNLRHMALDMGNEIDTQNRQIDRIMEKLIPIKPGLMKPTSVQQRCSAVVKCSKVHFLLMLSQRAVPSCFYHGIYLLGLHTCTYQPHCKCCPV

SEQ ID NO: 7 - protein, 118 aa, Homo sapiens
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGAICAIIVVVIVIYFFT

SEQ ID NO: 8 - protein, 116 aa, Homo sapiens
MSATAATAPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFSS

SEQ ID NO: 9 - protein, 116 aa, Mus musculus
MSATAATVPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 10 - protein, 116 aa, Bos taurus; identical to SEQ ID NO: 8

SEQ ID NO: 11 - protein, 114 aa, Xenopus laevis
MSAPAAGPPAAAPGDGAPQGPPNLTSNRRLQQTQAQVDEVVDIMRVNVDKVLERDTKLSELDDRADALQAGASQFETSAAKLKRKYWWKNMKMMIIMGVICAIILIIIIVYFST

SEQ ID NO: 12 - protein, 104 aa, Strongylocentrotus purpuratus
MAAPPPPQPAPSNKRLQQTQAQVDEVVDIMRVNVDKVLERDQALSVLDDRADALQQGASQFETNAGKLKRKYWWKNCKMMIILAIIIIVILIIIIVAIVQSQKK

SEQ ID NO: 13 - protein, 288 aa, Homo sapiens
MKDRTQELRTAKDSDDDDDVAVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTTSEELEDMLESGNPAIFASGIIMDSSISKQALSEIETRHSEIIKLENSIRELHDMFMDMAMLVESQGEMIDRIEYNVEHAVDYVERAVSDTKKAVKYQSKARRKKIMIIICCVILGIVIASTVGGIFA

SEQ ID NO: 14 - protein, 288 aa, Homo sapiens
MKDRTQELRSAKDSDDEEEVVHVDRDHFMDEFFEQVEEIRGCIEKLSEDVEQVKKQHSAILAAPNPDEKTKQELEDLTADIKKTANKVRSKLKAIEQSIEQEEGLNRSSADLRIRKTQHSTLSRKFVEVMTEYNATQSKYRDRCKDRIQRQLEITGRTTTNEELEDMLESGKLAIFTDDIKMDSQMTKQALNEIETRHNEIIKLETSIRELHDMFVDMAMLVESQGEMIDRIEYNVEHSVDYVERAVSDTKKAVKYQSKARRKKIMIIICCVVLGVVLASSIGGTLGL

SEQ ID NO: 15 - protein, 288 aa, Mus musculus
MKDRTQELRTAKDSDDDDDVTVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTTSEELEDMLESGNPAIFASGIIMDSSISKQALSEIETRHSEIIKLETSIRELHDMFMDMAMLVESQGEMIDRIEYNVEHAVDYVERAVSDTKKAVKYQSKARRKKIMIIICCVILGIIIASTIGGIFG

SEQ ID NO: 16 - protein, 291 aa, Drosophila sp.
MTKDRLAALHAAQSDDEEETEVAVNVDGHDSYMDDFFAQVEEIRGMIDKVQDNVEEVKKKHSAILSAPQTDEKTKQELEDLMADIKKNANRVRGKLKGIEQNIEQEEQQNKSSADLRIRKTQHSTLSRKFVEVMTEYNRTQTDYRERCKGRIQRQLEITGRPTNDDELEKMLEEGNSSVFTQGIIMETQQAKQTLADIEARHQDIMKLETSIKELHDMFMDMAMLVESQGEMIDRIEYHVEHAMDYVQTATQDTKKALKYQSKARRKKIMILICLTVLGILAASYVSSYFM

SEQ ID NO: 17 - protein, 291 aa, C. elegans
MTKDRLSALKAAQSEDEQDDDMHMDTGNAQYMEEFFEQVEEIRGSVDIIANNVEEVKKKHSAILSNPVNDQKTKEELDELMAVIKRAANKVRGKLKLIENAIDHDEQGAGNADLRIRKTQHSTLSRRFVEVMTDYNKTQTDYRERCKGRIQRQLDIAGKQVGDEDLEEMIESGNPGVFTQGIITDTQQAKQTLADIEARHNDIMKLESSIRELHDMFMDMAMLVESQGEMVDRIEYNVEHAKEFVDRAVADTKKAVQYQSKARRKKICILVTGVILITGLIIFILFYAKVL

SEQ ID NO: 18 - protein, 288 aa, Strongylocentrotus purpuratus
MRDRLGSLKRNEEDDVGPEVAVNVESEKFMEEFFEQVEEVRNNIDKISKNVDEVKKKHSDILSAPQADEKVKDELEELMSDIKKTANKVRAKLKMMEQSIEQEESAKMNSADVRIRKTQHSTLSRKFVEVMTDYNSTQTDYRERCKGRIQRQLEITGKSTTDAELEDMLESGNPAIFTSGIIMDTQQAKQTLRDIEARHNDIIKLESSIRELHDMFMDMAMLVESQGEMIDRIEYNVEQSVDYVETAKMDTKKAVKYQSKARRKKFYIAICCGVALGILVLVLIIVLA

SEQ ID NO: 19 - DNA, 1005 bp, Artificial Sequence
  Plasmid pQPIGFP-SNAP25
  CDS (1)...(1005)
atggctagcaaaggagaagaactcttcactggagttgtcccaattcttgttgaattagatggtgatgttaacggccacaagttctctgtcagtggagagggtgaaggtgatgcaacatacggaaaacttaccctgaagttcatctgcactactggcaaactgcctgttccatggccaacactagtcactactctgtgctatggtgttcaatgcttttcaagatacccggatcatatgaaacggcatgactttttcaagagtgccatgcccgaaggttatgtacaggaaaggaccatcttcttcaaagatgacggcaactacaagacacgtgctgaagtcaagtttgaaggtgatacccttgttaatagaatcgagttaaaaggtattgacttcaaggaagatggcaacattctgggacacaaattggaatacaactataactcacacaatgtatacatcatggcagacaaacaaaagaatggaatcaaagtgaacttcaagacccgccacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactgtacaacggcggtgcaggatccggtgcgggtggcggtggcatccggagggtaacaaacgatgcccgggaaaatgagatggatgagaacctggagcaggtgagcggcatcatcggaaacctccgccatatggctctagacatgggcaatgagattgacacccagaatcgccagatcgacaggatcatggagaaggctgattccaacaaaaccagaattgatgaagccaaccaacgtgcaacaaagatgctgggaagtggtggcggtggcggccatcaccatcaccatcactgctaa

SEQ ID NO: 20 - protein, 334 aa, Artificial Sequence
  Plasmid pQBI GFP-SNAP25
MASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNGGAGSGAGGGGIRRVTNDARENEMDENLEQVSGIIGNLRHMALDMGNEIDTQNRQIDRIMEKADSNKTRIDEANQRATKMLGSGGGGGHHHHHHC

SEQ ID NO: 21 - protein, 5 aa, Clostridium sp.
GGGGS

SEQ ID NO: 22 - protein, 8 aa, Artificial Sequence
  synthetic construct
EANQRATK

SEQ ID NO: 23 - protein, 8 aa, Artificial Sequence
  synthetic construct
GASQFETS

SEQ ID NO: 24 - protein, 8 aa, Artificial Sequence
  synthetic construct
DTKKAVKW

SEQ ID NO: 25 - protein, 8 aa, Artificial Sequence
  synthetic construct
RDQKLSEL

SEQ ID NO: 26 - protein, 8 aa, Artificial Sequence
  synthetic construct
QIDRIMEK

SEQ ID NO: 27 - protein, 8 aa, Artificial Sequence
  synthetic construct
ERDQKLSE

SEQ ID NO: 28 - protein, 8 aa, Artificial Sequence
  synthetic construct
ETSAAKLK

SEQ ID NO: 29 - protein, 8 aa, Artificial Sequence
  synthetic construct
GASQFETS

SEQ ID NO: 30 - protein, 13 aa, Homo sapiens
TRIDEANQRATKM

SEQ ID NO: 31 - protein, 15 aa, Homo sapiens
SNKTRIDEANQRATK

SEQ ID NO: 32 - protein, 16 aa, Homo sapiens
SNKTRIDEANQRATKM

SEQ ID NO: 33 - protein, 17 aa, Homo sapiens
SNKTRIDEANQRATKML

SEQ ID NO: 34 - protein, 17 aa, Homo sapiens
DSNKTRIDEANQRATKM

SEQ ID NO: 35 - protein, 18 aa, Homo sapiens
DSNKTRIDEANQRATKML

SEQ ID NO: 36 - protein, 33 aa, Mus musculus
QNRQIDRIMEKADSNKTRIDEANQRATKMLGSG

SEQ ID NO: 37 - protein, 32 aa, Homo sapiens
QNPQIKRITDKADTNRDRIDIANARAKKLIDS

SEQ ID NO: 38 - protein, 32 aa, Mus musculus
QNQQIQKITEKADTNKNRIDIANTRAKKLIDS

SEQ ID NO: 39 - protein, 34 aa, Gallus gallus
QNRQIDRIMEKLIPIKPGLMKPTSVQQRCSAVVK

SEQ ID NO: 40 - protein, 33 aa, Carassius auratus
QNRQIDRIMDMADSNKTRIDEANQRATKMLGSG

SEQ ID NO: 41 - protein, 33 aa, Carassius auratus; identical to SEQ ID NO: 36

SEQ ID NO: 42 - protein, 30 aa, Torpedo sp.
QNAQVDRIVVKGDMNKARIDEANKHATKML

SEQ ID NO: 43 - protein, 33 aa, Strongylocentrotus purpuratus
QNSQVGRITSKAESNEGRINSADKRAKNILRNK

SEQ ID NO: 44 - protein, 31 aa, C. elegans
QNRQLDRIHDKQSNEVRVESANKRAKNLITK

SEQ ID NO: 45 - protein, 31 aa, Drosophila sp.
QNRQIDRINRKGESNEARIAVANQRAHQLLK

SEQ ID NO: 46 - protein, 32 aa, Hirudinida sp.
QNRQVDRINNKMTSNQLRISDANKRASKLLKE

SEQ ID NO: 47-49 - skipped (3 entries)

SEQ ID NO: 50 - protein, 17 aa, Artificial Sequence
  synthetic construct
SNKTRIDEANQRATKAL

SEQ ID NO: 51 - protein, 17 aa, Artificial Sequence
  synthetic construct
  MOD_RES 16: Xaa=Nle
SNKTRIDEANQRATKXL

SEQ ID NO: 52 - protein, 17 aa, Artificial Sequence
  synthetic construct
SNKTRIDEANQRATAML

SEQ ID NO: 53 - protein, 17 aa, Artificial Sequence
  synthetic construct
SNKTRIDEANQRASKML

SEQ ID NO: 54 - protein, 17 aa, Artificial Sequence
  synthetic construct
  MOD_RES 14: Xaa=Abu
SNKTRIDEANQRAXKML

SEQ ID NO: 55 - protein, 17 aa, Artificial Sequence
  synthetic construct
  MOD_RES 13: Xaa=Abu
SNKTRIDEANQRXTKML

SEQ ID NO: 56 - protein, 17 aa, Artificial Sequence
  synthetic construct
SNKTRIDEANARATKML

SEQ ID NO: 57 - protein, 17 aa, Artificial Sequence
  synthetic construct
  MOD_RES 11: Xaa=Abu
SNKTRIDEANXRATKML

SEQ ID NO: 58 - protein, 17 aa, Artificial Sequence
  synthetic construct
SNKTRIDEANNRATKML

SEQ ID NO: 59 - protein, 17 aa, Artificial Sequence
  synthetic construct
SNKTRIDEAAQRATKML

SEQ ID NO: 60 - protein, 17 aa, Artificial Sequence
  synthetic construct
  MOD_RES 9: Xaa=Abu
SNKTRIDEXNQRATKML

SEQ ID NO: 61 - protein, 17 aa, Artificial Sequence
  synthetic construct
SNKTRIDQANQRATKML

SEQ ID NO: 62 - protein, 17 aa, Artificial Sequence
  synthetic construct
SNKTRINEANQRATKML

SEQ ID NO: 63 - protein, 40 aa, Homo sapiens
DKVLERDQKLSELDDRADALQAGASQFESSAAKLKRKYWW

SEQ ID NO: 64 - protein, 40 aa, Bos taurus
DKVLERDQKLSELDDRADALQAGASQFETSAAKLKRKYWW

SEQ ID NO: 65 - protein, 40 aa, Rattus sp.
DKVLERDQKLSELDDRADALQAGASVFESSAAKLKRKYWW

SEQ ID NO: 66 - protein, 40 aa, Rattus sp.; identical to SEQ ID NO: 64

SEQ ID NO: 67 - protein, 40 aa, Rattus sp.; identical to SEQ ID NO: 64

SEQ ID NO: 68 - protein, 40 aa, Rattus sp.
DLVAQRGERLELLIDKTENLVDSSVTFKTTSRNLARAMCM

SEQ ID NO: 69 - protein, 32 aa, Gallus gallus
ERDQKLSELDDRADALQAGASVFESSAAKLKR

SEQ ID NO: 70 - protein, 32 aa, Gallus gallus
ERDQKLSELDDRADALQAGASQFETSAAKLKR

SEQ ID NO: 71 - protein, 40 aa, Torpedo sp.; identical to SEQ ID NO: 63

SEQ ID NO: 72 - protein, 40 aa, Strongylocentrotus purpuratus
DKVLDRDGALSVLDDRADALQQGASQFETNAGKLKRKYWW

SEQ ID NO: 73 - protein, 40 aa, Aplysia sp.
EKVLDRDQKISQLDDRAEALQAGASQFEASAGKLKRKYWW

SEQ ID NO: 74 - protein, 40 aa, Teuthoida sp.
DKVLERDSKISELDDRADALQAGASQFEASAGKLKRKFWW

SEQ ID NO: 75 - protein, 40 aa, C. elegans
NKVMERDVQLNSLDHRAEVLQNGASQFQQSSRELKRQYWW

SEQ ID NO: 76 - protein, 40 aa, Drosophila melanogaster
EKVLERDQKLSELGERADQLEGGASQSEQQAGKLKRKQWW

SEQ ID NO: 77 - protein, 40 aa, Drosophila melanogaster
EKVLERDSKLSELDDRADALQQGASQFEQQAGKLKRKFWL

SEQ ID NO: 78 - protein, 39 aa, Hirudinida sp.
DKVLEKDQKLAELDRADALQAGASQFEASAGKLKRKFWW

SEQ ID NO: 79 - protein, 18 aa, Homo sapiens
ERAVSDTKKAVKYQSKAR

SEQ ID NO: 80 - protein, 18 aa, Bos taurus
ERAVSDTKKAVKYQSKAR

SEQ ID NO: 81 - protein, 18 aa, Rattus rattus
EHAKEETKKAIKYQSKAR

SEQ ID NO: 82 - protein, 18 aa, Rattus rattus
EKARDETRKAMKYQGGAR

SEQ ID NO: 83 - protein, 18 aa, Rattus rattus
ERGQEHVKIALENQKKAR

SEQ ID NO: 84 - protein, 18 aa, Gallus gallus
VPEVFVTKSAVMYQCKSR

SEQ ID NO: 85 - protein, 18 aa, Strongylocentrotus purpuratus
VRRQNDTKKAVKYQSKAR

SEQ ID NO: 86 - protein, 18 aa, Aplysia sp.
ETAKMDTKKAVKYQSKAR

SEQ ID NO: 87 - protein, 18 aa, Teuthoida sp.
ETAKVDTKKAVKYQSKAR

SEQ ID NO: 88 - protein, 18 aa, Drosophila melanogaster
QTATQDTKKALKYQSKAR

SEQ ID NO: 89 - protein, 18 aa, Hirudinida sp.
ETAAADTKKAMKYQSAAR

SEQ ID NO: 90 - protein, 116 aa, Rattus sp.; identical to SEQ ID NO: 9

SEQ ID NO: 91 - DNA, 36 bp, Artificial Sequence
  primer
gctagatctcgagttaaccacttcccagcatctttg

SEQ ID NO: 92 - DNA, 24 bp, Artificial Sequence
  primer
atccggagggtaacaaacgatgcc

SEQ ID NO: 93 - DNA, 60 bp, Artificial Sequence
  primer
cgaattccgcgggccaccatgggaggaggactgaacgacatcttcgaggctcaaaagatc

SEQ ID NO: 94 - DNA, 79 bp, Artificial Sequence
  primer
tcgtttgttaccctccggatatgatgatgatgatgatgatgatgggatccatgccactcgatcttttgagcctcgaaga

SEQ ID NO: 95 - DNA, 30 bp, Artificial Sequence
  primer
cgaagatctggaggactgaacgacatcttc

SEQ ID NO: 96 - DNA, 34 bp, Artificial Sequence
  primer
gatggtgatggtgatgacagccgccaccgccacc

SEQ ID NO: 97 - protein, 14 aa, Artificial Sequence
  synthetic peptide
GDKNADGWIEFEEL

SEQ ID NO: 98 - protein, 14 aa, Artificial Sequence
  synthetic peptide
GDKNADGFICFEEL

SEQ ID NO: 99 - protein, 12 aa, Artificial Sequence
  synthetic peptide
DKNADGCIEFEE

SEQ ID NO: 100 - protein, 17 aa, Artificial Sequence
  synthetic peptide
YIDTNNDGWYEGDELLA

SEQ ID NO: 101 - protein, 32 aa, Artificial Sequence
  synthetic peptide
TERRQQLDKDGDGTIDEREIKIWFQNKRAKIK